(19) United States
(12) Patent Application Publication
(10) Pub. No.: US 2024/0342492 A1
(43) Pub. Date: Oct. 17, 2024

(54) IMPLANT-TO-IMPLANT (I2I) COMMUNICATION

(71) Applicant: Pacesetter, Inc., Sylmar, CA (US)

(72) Inventors: Weiqun Yang, Cupertino, CA (US); Fady Dawoud, Studio City, CA (US)

(73) Assignee: Pacesetter, Inc., Sylmar, CA (US)

(21) Appl. No.: 18/629,725

(22) Filed: Apr. 8, 2024

Related U.S. Application Data
(60) Provisional application No. 63/495,717, filed on Apr. 12, 2023.

(51) Int. Cl. A61N 1/372; A61N 1/05; A61N 1/37; A61N 1/375

(52) U.S. Cl. CPC A61N 1/37288; A61N 1/3706; A61N 1/3756; A61N 1/0573

(57) ABSTRACT

Systems and methods for improving conducted i2i communication between first and second implantable medical device (IMDs) are described, wherein at least one of the IMDs comprises a leadless pacemaker (LP). Respective information is stored within each of the first and second IMDs that specifies a primary communication window (PCW) for performing the conducted communication with one another when using a primary conducted communication protocol (PCCP), and also specifies an alternate communication window (ACW) for performing the conducted communication with one another when using an alternate conducted communication protocol (ACCP). At least one of the first and second IMDs monitors quality metric of the conducted communication therebetween while the PCCP is being used, and in response to the quality metric falling below a corresponding threshold, the first and second IMDs perform conducted i2i communication with one another in accordance with the ACCP during the ACW of one or more cardiac cycles.

### PRIORITY CLAIM

This application claims priority to U.S. Provisional Patent Application No. 63/495,717, filed Apr. 12, 2023, which is incorporated herein by reference.

### RELATED APPLICATIONS

This application is related to U.S. patent application Ser. No. 18/162,226, filed Jan. 31, 2023, which is a continuation of U.S. patent application Ser. No. 17/370,210, filed Jul. 8, 2021, which is a continuation of U.S. patent application Ser. No. 16/171,080, filed Oct. 25, 2018 (now issued as U.S. Pat. No. 11,090,497).

### FIELD OF TECHNOLOGY

Embodiments described herein generally relate to methods and systems for improving communication between implantable medical devices, such as, but not limited to, leadless pacemakers.

### BACKGROUND

Some medical systems rely on wireless communication between multiple implantable medical devices (IMDs). For example, in certain cardiac pacing systems, multiple IMDs wirelessly communicate with one another to reliably and safely coordinate pacing and/or sensing operations. Such a system may include, for example, one or more leadless pacemakers (LPs), an implantable cardioverter-defibrillator (ICD), such as a subcutaneous-ICD, and/or an external programmer or other type of external device (e.g., a bedside monitor or other remote monitor) that is intended to wirelessly communicate with one or more IMDs. For a more specific example, certain such systems include an LP in the right ventricle (RV) and another LP in the right atrium (RA), wherein the LPs in the RV and RA wirelessly communicate with one another to coordinate pacing and/or sensing operations. Such wireless communication between two IMDs (e.g., two LPs) is often referred to as implant-to-implant (i2i) communication.

When using a pair of LPs to perform pacing and/or sensing operations in the RA and RV, one of the challenges is that i2i communication is relied upon to maintain appropriate synchrony between the RA and the RV. However, it has been observed that such i2i communication can be adversely affected by the orientation of the LPs relative to one another, especially where the i2i communication is conducted.

### SUMMARY

Certain embodiments of the present technology are directed to systems including first and second implantable medical devices (IMDs) that are configured to perform conducted communication with one another, wherein at least one of the first and second IMDs comprises a leadless pacemaker (LP) configured to be implanted within or on a cardiac chamber. Other embodiments of the present technology are directed to methods for use with such a system. In accordance with certain embodiments, such a method comprises the first and second IMDs performing the conducted communication with one another during a primary communication window (PCW) of one or more cardiac cycles in accordance with a primary conducted communication protocol (PCCP). The method also includes at least one of the first and second IMDs monitoring a quality metric of the conducted communication, while the first and second IMDs are performing the conducted communication with one another in accordance with the PCCP. Additionally, the method includes, in response to the quality metric of the conducted communication falling below a corresponding threshold (e.g., for at least a specified amount of time, for at least a specified number of cardiac cycles, or for at least X out of Y cardiac cycles), the first and second IMDs performing the conducted communication with one another during an alternative communication window (ACW) of one or more further cardiac cycles in accordance with an alternative conducted communication protocol (ACCP).

In accordance with certain embodiments, the method further includes storing respective information within each of the first and second IMDs that specifies the PCW for performing the conducted communication with one another when using the PCCP, and also specifies the ACW for performing the conducted communication with one another when using the ACCP.

In accordance with certain embodiments, the method further includes, over a plurality of different cardiac cycles, testing a plurality of different windows for performing conducted communication between the first and second LPs to thereby identify the ACW.

In accordance with certain embodiments, a distance between the first and second IMDs and an orientation of the first and second IMDs relative to one another vary over a cardiac cycle. In certain such embodiments, the ACW corresponds to a period within the cardiac cycle when the distance between the first and second IMDs and the orientation of the first and second IMDs relative to one another are such that it is likely that the conducted communication between the first and second IMDs will be successful.

In accordance with certain embodiments, when the first and second IMDs perform conducted communication with one another using the PCCP, the first IMD transmits one or more conducted communication pulses to the second IMD within the PCW that occurs immediately prior to a paced event caused by the first IMD or immediately following a sensed event sensed by the first IMD.

In accordance with certain embodiments, when the first and second IMDs perform conducted communication with one another during the ACW using the ACCP, the first and second IMDs are also attempting to perform the conducted communication with one another during the PCW.

In accordance with certain embodiments, when the first and second IMDs perform conducted communication with one another during the ACW using the ACCP, the first and second IMDs do not attempt to perform conducted communication with one another during the PCW.

In accordance with certain embodiments, performing conducted communication comprises one of the first and second IMDs transmitting one or more conducted communication pulses to the other one of the first and second IMDs, and the other one of the first and second IMDs attempting to receive the one or more conducted communication pulses. In certain such embodiments, the quality metric is based on one or more of the following: a measure of amplitude of at least one of the one or more conducted communication pulses received by one of the first and second IMDs; a magnitude of at least one of the one or more conducted communication pulses received by one of the first and second IMDs; a measure of signal throughput; a measure of signal loss in at least one of the one or more conducted communication pulses received by one of the first and second IMDs; a signal-to-noise ratio (SNR) of at least one of the one or more conducted communication pulses received by one of the first and second IMDs; a total energy of at one of the one or more conducted communication pulses received by one of the first and second IMDs; or a bit-error-rate (BER) associated with at least one of the one or more conducted communication pulses received by one of the first and second IMDs.

In accordance with certain embodiments, the first and second IMDs comprise first and second LPs. In certain such embodiments, one of the first and second LPs is configured to be implanted in or on an atrial cardiac chamber, and the other one of the first and second LPs is configured to be implanted in or on a ventricular cardiac chamber. Alternatively, one of the first and second LPs is configured to be implanted in or on a right ventricular cardiac chamber, and the other one of the first and second LPs is configured to be implanted in or on a left ventricular cardiac chamber. Other variations are also possible and within the scope of the embodiments described herein.

In accordance with certain embodiments the method further includes, in response to the quality metric of the conducted communication falling below the corresponding threshold, at least one of the IMDs selecting the ACW from a plurality of possible ACWs based on a posture of a patient within which the first and second IMDs are implanted.

Certain embodiments of the present technology are directed to an implantable system comprising first and second IMDs, at least one of which comprises an LP configured to be implanted in or on a first cardiac chamber. Each IMD, of the first and second IMDs, comprising a respective controller, a respective memory, one or more respective sense circuits, one or more respective pulse generators, and a respective pair of electrodes. The one or more pulse generators and the pair of electrodes, of at least the LP, is/are configured to output pacing pulses and output conducted communication pulses. The one or more sense circuits and the pair of electrodes, of each IMD, of the first and second IMDs, is/are configured to sense a cardiac electrical signal and sense conducted communication pulses. The controller of each IMD, of the first and second IMDs, is communicatively coupled to the memory, the one or more sense circuits, and the one or more pulse generators of the IMD, and is configured to perform conducted communication with the other IMD during a primary communication window (PCW) of one or more cardiac cycles in accordance with a primary conducted communication protocol (PCCP). The controller of at least one of the first and second IMDs is configured to monitor a quality metric of the conducted communication, while the first and second IMDs are performing the conducted communication with one another in accordance with the PCCP. In response to the quality metric of the conducted communication falling below a corresponding threshold (e.g., for at least a specified amount of time, for at least a specified number of cardiac cycles, or for at least X out of Y cardiac cycles), the controller is configured to cause the conducted communication to be performed during an alternative communication window (ACW) of one or more further cardiac cycles in accordance with an alternative conducted communication protocol (ACCP).

In accordance with certain embodiments, whenever a critical message is sent from one of the first and second IMDs to the other using conducted communication, the critical message is sent during both the PCW and the ACW of a same cardiac cycle to thereby increase a probability that the critical message will be successful received.

In accordance with certain embodiments, the memory, of each IMD, of the first and second IMDs, stores information that specifies the PCW for performing conducted communication using the PCCP, and also specifies the ACW for performing the conducted communication using the ACCP.

In accordance with certain embodiments, a distance between the first and second IMDs and an orientation of the first and second IMDs relative to one another vary over a cardiac cycle. In certain such embodiments, the ACW corresponds to a period within the cardiac cycle when the distance between the first and second IMDs and the orientation of the first and second IMDs relative to one another are such that it is likely that the conducted communication between the first and second IMDs will be successful.

In accordance with certain embodiments, a plurality of different windows for performing conducted communication between the first and second IMDs are tested to thereby identify the ACW.

In accordance with certain embodiments, when the first and second IMDs perform conducted communication with one another using the PCCP, the first IMD transmits one or more conducted communication pulses to the second IMD within the PCW that occurs immediately prior to a paced event caused by the first IMD or immediately following a sensed event sensed by the first IMD.

In accordance with certain embodiments, when the first and second IMDs perform conducted communication with one another during the ACW using the ACCP, the first and second IMDs are also attempting to perform the conducted communication with one another during the PCW.

In accordance with certain embodiments, when the first and second IMDs perform conducted communication with one another using the ACCP, the first and second IMDs do not attempt to perform conducted communication with one another during the PCW.

In accordance with certain embodiments, the first and second IMDs of the system comprise first and second LPs. In certain such embodiments, one of the first and second LPs is configured to be implanted in or on an atrial cardiac chamber, and the other one of the first and second LPs is configured to be implanted in or on a ventricular cardiac chamber. Alternatively, one of the first and second LPs is configured to be implanted in or on a right ventricular cardiac chamber, and the other one of the first and second LPs is configured to be implanted in or on a left ventricular cardiac chamber. Other variations are also possible and within the scope of the embodiments described herein.

In accordance with certain embodiments, the controller of at least one of the first and second IMDs is configured such that whenever a critical message is sent from one of the first and second IMDs to the other using conducted communication, the critical message is sent during both the PCW and the ACW of a same cardiac cycle to thereby increase a probability that the critical message will be successful received.

In accordance with certain embodiments, the controller of at least one of the first and second IMDs is configured to, in response to the quality metric of the conducted communication falling below the corresponding threshold, select the ACW from a plurality of possible ACWs based on a posture of a patient within which the first and second IMDs are implanted.

A method according to another embodiment of the present technology comprises testing a plurality of different communication windows (CWs) for performing conducted communication between first and second IMDs, for each of a plurality of different postures of the patient, to thereby perform the testing for each of a plurality of different combinations of the different postures and the different CWs, wherein at least one of the first and second IMDs comprises an LP implanted in or on a cardiac chamber. The method also includes determining and storing information indicative of a respective communication quality metric for each of the plurality of different combinations of the different postures and the different CWs. The method additionally includes selecting one of the CWs to use for performing the conducted communication based on the stored information, and performing the conducted communication between the first and second IMDs using the selected one of the CWs. In certain such embodiments, the selecting comprises selecting the one of the CWs that, based on the stored information, should result in the conducted communication being successful regardless of which one of the plurality of different postures the patient is positioned. In accordance with certain embodiments, each of the plurality of CWs corresponds to a different delay relative to at least one of a paced or sensed event.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

### BRIEF DESCRIPTION OF THE DRAWINGS

Embodiments of the present technology relating to both structure and method of operation may best be understood by referring to the following description and accompanying drawings, in which similar reference characters denote similar elements throughout the several views:

FIG. 1A illustrates a system formed in accordance with certain embodiments described herein as implanted in a heart.

FIG. 1B is a block diagram of an LP in accordance with certain embodiments herein.

FIG. 2 illustrates an LP in accordance with certain embodiments herein.

FIG. 3 is a timing diagram demonstrating one embodiment of conducted i2i communication for a paced event.

FIG. 4 is a timing diagram demonstrating one embodiment of conducted i2i communication for a sensed event.

FIG. 5 is a diagram that is used to show how the orientation of two different LPs can be quantified in accordance with certain embodiments of the present technology.

FIG. 6 is a high level flow diagram that is used to summarize certain embodiments of the present technology that enable LPs to selectively transition between performing conducted i2i communication with one another within PCWs in accordance with a PCCP, and performing conducted communication with one another within ACWs in accordance with an ACCP.

FIGS. 7A and 7B are example timing diagrams showing how LPs can communicate with one another using conducted i2i communication within PCWs in accordance with the PCCP.

FIGS. 7C and 7D are example timing diagrams showing how LPs can communicate with one another using conducted i2i communication within ACWs in accordance with the ACCP.

FIG. 8 is a high level flow diagram used to describe a technique for identifying the ACW that is to be used when the ACCP is used, in accordance with certain embodiments of the present technology.

FIG. 9 is a high level flow diagram used to describe a technique for identifying a CW that can be used for conducted i2i communication while the patient is in a plurality of different postures, in accordance with certain embodiments of the present technology.

FIGS. 10A and 10B illustrate example of distances between and orientation angels between an atrial LP (aLP) and a ventricular LP (vLP) at two different times during a cardiac cycle, while the patient is in the same orientation. FIG. 10C is a graph that plots distances and angles over a cardiac cycle, to thereby show how the distances and orientation of an aLP and a vLP may vary relative to one another during a cardiac cycle.

FIG. 11 shows a block diagram of one embodiment of an LP (or other type of IMD) that is implanted into a patient as part of an implantable cardiac system in accordance with certain embodiments herein.

### DETAILED DESCRIPTION

Certain embodiments of the present technology relate to implantable systems, and methods for use therewith, that improve conducted i2i communication between multiple implantable medical device (IMDs), such as between a pair of leadless pacemakers (LPs), or between a leadless pacemaker (LP) and an implantable cardioverter-defibrillator (ICD), or between an LP and an insertable cardiac monitor (ICM), but not limited thereto.

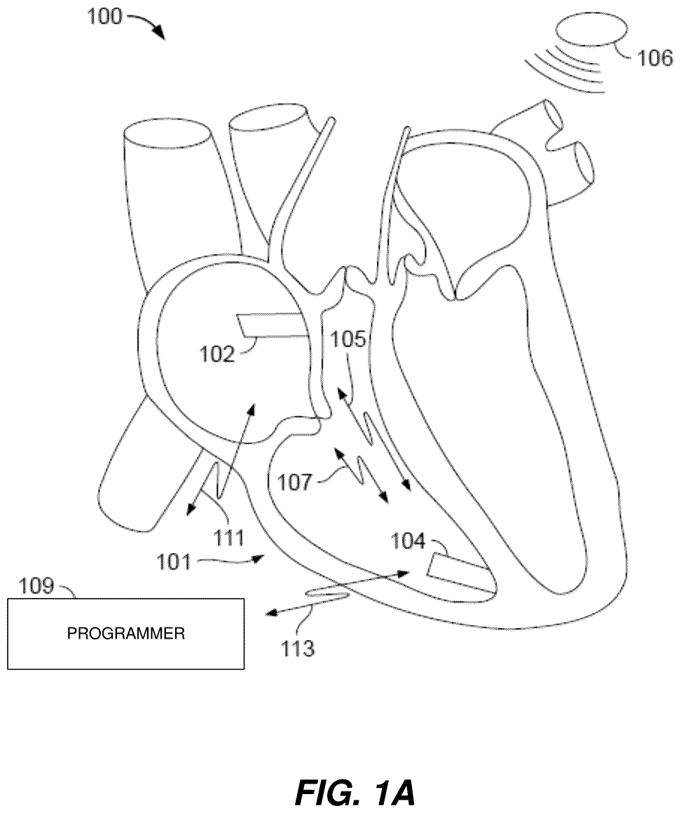

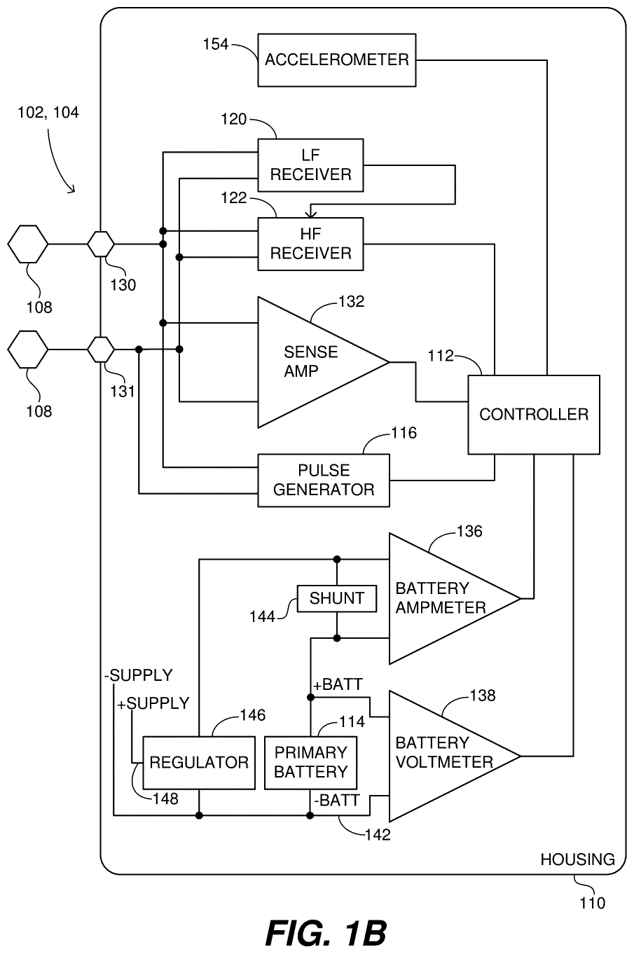

Before providing addition details of the specific embodiments of the present technology mentioned above, an exemplary system in which embodiments of the present technology can be used will first be described with reference to FIGS. 1A, 1B and 2. More specifically, FIGS. 1A, 1B and 2 will be used to describe an example cardiac pacing system, wherein pacing and sensing operations can be performed by multiple medical devices, which may include one or more LPs, an ICD, such as a subcutaneous-ICD, and/or a programmer reliably and safely coordinate pacing and/or sensing operations.

FIG. 1A illustrates a system 100 formed in accordance with certain embodiments herein as implanted in a heart 101. The system 100 comprises two or more LPs 102 and 104 located in different chambers of the heart. The LP 102 is located in a right atrium (RA), while the LP 104 is located in a right ventricle (RV). The RA is also known as the right atrial chamber, and the RV is also known as the right ventricular chamber. Such chambers of a patient's heart can also be referred to herein as cardiac chambers. LPs 102 and 104 communicate with one another to inform one another of various local physiologic activities, such as local intrinsic events, local paced events and the like. LPs 102 and 104 may be constructed in a similar manner, but operate differently based upon which chamber LP 102 or 104 is located.

In some embodiments, LPs 102 and 104 communicate with one another, with an ICD 106, and with an external device (e.g., programmer) 109 through wireless transceivers, communication coils and antenna, and/or by conducted i2i communication through the same electrodes as used for sensing and/or delivery of pacing therapy. When conducted i2i communication is maintained through the same electrodes as used for pacing, the system 100 may omit an antenna or telemetry coil in one or more of LPs 102 and 104.

In some embodiments, one or more LPs 102 and 104 can be co-implanted with the ICD 106. Each LP 102, 104 uses two or more electrodes located within, on, or within a few centimeters of the housing of the LP, for pacing and sensing at the cardiac chamber, for bidirectional communication with one another, with the external device (e.g., programmer) 109, and the ICD 106.

In accordance with certain embodiments, methods are provided for coordinating operation between LPs located in different chambers of the heart. The methods can configure a local LP to receive communication from a remote LP through conducted i2i communication. While the methods and systems described herein include examples primarily in the context of LPs, it is understood that the methods and systems herein may be utilized with various other IMDs. By way of example, the methods and systems may coordinate operation between various other types implantable medical devices (IMDs) implanted in a human, not just LPs.

Referring to FIG. 1B, a block diagram shows exemplary electronics within LPs 102 and 104. LP 102, 104 includes first and second receivers 120 and 122 that collectively define separate first and second communication channels 105 and 107 (FIG. 1A), (among other things) between LPs 102 and 104. Although first and second receivers 120 and 122 are depicted, in other embodiments, LP 102, 104 may only include first receiver 120, or may include additional receivers other than first and second receivers 120 and 122. As will be described in additional detail below, the pulse generator 116 can function as a transmitter that transmits i2i communication signals using the electrodes 108. Usage of the electrodes 108 for communication enables the one or more LPs 102 and 104 to perform antenna-less and telemetry coil-less communication. A same pulse generator 116 can be used to produce both pacing pulses and conducted communication pulses, in which case an LP may only include a single pulse generator 116. Alternatively, an LP can include more than one pulse generator, e.g., one of which can be used to produce pacing pulses, and the other can be used to produce conducted communication pulses.

In accordance with certain embodiments, when one of the LPs 102 and 104 senses an intrinsic event or delivers a paced event, the corresponding LP 102, 104 transmits an implant event message to the other LP 102, 104. For example, when an atrial LP 102 senses/paces an atrial event, the atrial LP 102 transmits an implant event message including an event marker indicative of a nature of the event (e.g., intrinsic/sensed atrial event, paced atrial event). When a ventricular LP 104 senses/paces a ventricular event, the ventricular LP 104 transmits an implant event message including an event marker indicative of a nature of the event (e.g., intrinsic/sensed ventricular event, paced ventricular event). In certain embodiments, LP 102, 104 transmits an implant event message to the other LP 102, 104 preceding the actual pace pulse so that the remote LP can blank its sense inputs in anticipation of that remote pace pulse (to prevent inappropriate crosstalk sensing).

Still referring to FIG. 1B, each LP 102, 104 is shown as including a controller 112 and a pulse generator 116. The controller 112 can include, e.g., a microprocessor (or equivalent control circuitry), RAM and/or ROM memory, logic and timing circuitry, state machine circuitry, and I/O circuitry, but is not limited thereto. The controller 112 can further include, e.g., timing control circuitry to control the timing of the stimulation pulses (e.g., pacing rate, atrio-ventricular (AV) delay, atrial interconduction (A-A) delay, or ventricular interconduction (V-V) delay, etc.). Such timing control circuitry may also be used for the timing of refractory periods, blanking intervals, noise detection windows, evoked response windows, alert intervals, marker channel timing, and so on. The controller 112 can further include other dedicated circuitry and/or firmware/software components that assist in monitoring various conditions of the patient's heart and managing pacing therapies. The controller 112 and the pulse generator 116 may be configured to transmit event messages, via the electrodes 108, in a manner that does not inadvertently capture the heart in the chamber where LP 102, 104 is located, such as when the associated chamber is in a refractory state. In addition, an LP 102, 104 that receives an event message may enter an “event refractory” state (or event blanking state) following receipt of the event message. The event refractory/blanking state may be set to extend for a determined period of time after receipt of an event message in order to avoid the receiving LP 102, 104 from inadvertently sensing another signal as an event message that might otherwise cause retriggering. For example, the receiving LP 102, 104 may detect a measurement pulse from another LP 102, 104 or programmer 109.

In accordance with certain embodiments herein, the programmer 109 may communicate over a programmer-to-LP channel, with LP 102, 104 utilizing the same communication scheme. The external programmer 109 may listen to the event message transmitted between LP 102, 104 and synchronize programmer to implant communication such that programmer 109 does not transmit communication signals 113 until after an implant-to-implant messaging sequence is completed.

In some embodiments, the individual LP 102 (or 104) can comprise a hermetic housing 110 configured for placement on or attachment to the inside or outside of a cardiac chamber and at least two leadless electrodes 108 proximal to the housing 110 and configured for bidirectional communication with at least one other device 106 within or outside the body.

Referring to FIG. 1B, the LP 102 (or 104) is shown as optionally including an accelerometer 154 which can be hermetically contained within the housing 110. The accelerometer 154 can be any one of various different types of well-known accelerometers, or can be a future developed accelerometer. For one example, the accelerometer 154 can be or include, e.g., a MEMS (micro-electromechanical system) multi-axis accelerometer of the type exploiting capacitive or optical cantilever beam techniques, or a piezoelectric accelerometer that employs the piezoelectric effect of certain materials to measure dynamic changes in mechanical variables. Where the accelerometer is a multi-axis accelerometer it can include two or three sensors aligned along orthogonal axes. The accelerometer 154 can be, e.g., a one-dimensional (1D) accelerometer (also known as a one-axis accelerometer), a two-dimensional (2D) accelerometer (also known as a two-axis accelerometer), or a three-dimensional (3D) accelerometer (also known as a three-axis accelerometer). The output(s) of the accelerometer can be used to determine the orientation of the IMD, and thus, it can be said that the output(s) of the accelerometer (e.g., 154) are indicative of an orientation of the IMD (e.g., LP 102 or 104). More specifically, in accordance with certain embodiments, the controller 112 of an LP 102 (or 104) receives one or more outputs output(s) of the accelerometer 154, which is/are indicative of an orientation of the LP 102 (or 104). In such embodiments, the controller 112 can determine, based on the output(s) received from the accelerometer 154, an actual orientation of the LP 102 (or 104). Each output of the accelerometer 154 can comprise a respective signal.

One or more signals produced and output by the accelerometer 154 may be analyzed with respect to frequency content, energy, duration, amplitude and/or other characteristics. Such signals may or may not be amplified and/or filtered prior to being analyzed. For example, filtering may be performed using lowpass, highpass and/or bandpass filters. The signals output by the accelerometer 154 can be analog signals, which can be analyzed in the analog domain, or can be converted to digital signals (by an analog-to-digital converter) and analyzed in the digital domain. Alternatively, the signals output by the accelerometer 154 can already be in the digital domain. The one or more signals output by the accelerometer 154 can be analyzed by the controller 112 and/or other circuitry. In certain embodiments, the accelerometer 154 is packaged along with an integrated circuit (IC) that is designed to analyze the signal(s) it generates. In such embodiments, one or more outputs of the packaged sensor/IC can be an indication of acceleration along one or more axes. In other embodiments, the accelerometer 154 can be packaged along with an IC that performs signal conditioning (e.g., amplification and/or filtering), performs analog-to-digital conversions, and stores digital data (indicative of the sensor output) in memory (e.g., RAM, which may or may not be within the same package). In such embodiments, the controller 112 or other circuitry can read the digital data from the memory and analyze the digital data. Other variations are also possible, and within the scope of embodiments of the present technology. In accordance with certain embodiments, the LPs and/or other IMDs are devoid of an accelerometer 154.

FIG. 1B depicts a single LP 102 (or 104) and shows the LP's functional elements substantially enclosed in a hermetic housing 110. The LP 102 (or 104) has at least two electrodes 108 located within, on, or near the housing 110, for delivering pacing pulses to and sensing electrical activity from the muscle of the cardiac chamber, for sensing motion, for sensing temperature, and for bidirectional conducted communication with at least one other device within or outside the body. Hermetic feedthroughs 130, 131 conduct electrode signals through the housing 110. The housing 110 contains a primary battery 114 to supply power for pacing, sensing, and communication. The housing 110 also contains circuits 132 for sensing cardiac activity from the electrodes 108, receivers 120, 122 for receiving information from at least one other device via the electrodes 108, and the pulse generator 116 for generating pacing pulses for delivery via the electrodes 108 and also for transmitting information to at least one other device via the electrodes 108. The housing 110 can further contain circuits for monitoring device health, for example a battery current monitor 136 and a battery voltage monitor 138, and can contain circuits for controlling operations in a predetermined manner.

The receivers 120 and 122 can also be referred to, respectively, as a low frequency (LF) receiver 120 and a high frequency (HF) receiver 122, because the receiver 120 is configured to monitor for one or more signals within a relatively low frequency range (e.g., below 100 KHz) and the receiver 122 is configured to monitor for one or more signals within a relatively high frequency range (e.g., above 100 KHz). In certain embodiments, the receiver 120 (and more specifically, at least a portion thereof) is always enabled and monitoring for a wakeup notice, which can simply be a wakeup pulse, within a specific low frequency range (e.g., between 1 KHz and 100 KHz); and the receiver 122 is selectively enabled by the receiver 120. The receiver 120 is configured to consume less power than the receiver 122 when both the first and second receivers are enabled. Accordingly, the receiver 120 can also be referred to as a low power receiver 120, and the receiver 122 can also be referred to as a high power receiver 122. The low power receiver 120 is incapable of receiving signals within the relatively high frequency range (e.g., above 100 KHz), but consumes significantly less power than the high power receiver 122. This way the low power receiver 120 is capable of always monitoring for a wakeup notice without significantly depleting the battery (e.g., 114) of the LP. In accordance with certain embodiments, the high power receiver 122 is selectively enabled by the low power receiver 120, in response to the low power receiver 120 receiving a wakeup notice, so that the high power receiver 122 can receive the higher frequency signals, and thereby handle higher data throughput needed for effective i2i communication without unnecessarily and rapidly depleting the battery of the LP (which the high power receiver 122 may do if it were always enabled).

Since the receivers 120, 122 are used to receive conducted communication messages, the receivers 120, 122 can also be referred to as conducted communication receivers. In certain embodiments, each of the LPs 102 includes only a single conducted communication receiver. An example of a single conducted communication receiver, that can be included in the LPs and/or other types of IMDs referred to herein, is described in commonly assigned U.S. patent application Ser. No. 17/538,827, titled “Fully-Differential Receiver for Receiving Conducted Communication Signals,” filed Dec. 30, 2021, which is incorporated herein by reference.

The electrodes 108 can be configured to communicate bidirectionally among the multiple LPs and/or the implanted ICD 106 to coordinate pacing pulse delivery and optionally other therapeutic or diagnostic features using messages that identify an event at an individual LP originating the message and an LP receiving the message react as directed by the message depending on the origin of the message. An LP 102, 104 that receives the event message reacts as directed by the event message depending on the message origin or location. In some embodiments or conditions, the two or more leadless electrodes 108 can be configured to communicate bidirectionally among the one or more LPs 102, 104 and/or the ICD 106 and transmit data including designated codes for events detected or created by an individual LP. Individual LPs can be configured to issue a unique code corresponding to an event type and a location of the sending pacemaker.

Moreover, information communicated on the incoming channel can also include an event message from another leadless cardiac pacemaker signifying that the other leadless cardiac pacemaker has sensed a heartbeat or has delivered a pacing pulse, and identifies the location of the other pacemaker. For example, LP 104 may receive and relay an event message from LP 102 to the programmer. Similarly, information communicated on the outgoing channel can also include a message to another LP, or to the ICD, that the sending leadless cardiac pacemaker has sensed a heartbeat or has delivered a pacing pulse at the location of the sending pacemaker.

Referring again to FIGS. 1 and 2, the cardiac pacing system 100 may comprise an ICD 106 in addition to LPs 102, 104 configured for implantation in electrical contact with a cardiac chamber and for performing cardiac rhythm management functions in combination with the implantable ICD 106. The implantable ICD 106 and the one or more LPs 102, 104 can be configured for leadless intercommunication by information conduction through body tissue for wireless transmission between transmitters and receivers in accordance with the discussion herein.

As shown in the illustrative embodiments, an LP 102, 104 can comprise two or more leadless electrodes 108 configured for delivering cardiac pacing pulses, sensing evoked and/or natural cardiac electrical signals, and bidirectionally communicating with the co-implanted ICD 106.

LP 102, 104 can be configured for operation in a particular location and a particular functionality at manufacture and/or at programming by an external programmer 109. Bidirectional communication among the multiple LPs can be arranged to communicate notification of a sensed heartbeat or delivered pacing pulse event and encoding type and location of the event to another implanted pacemaker or pacemakers. LP 102, 104 receiving the communication decode the information and respond depending on location of the receiving pacemaker and predetermined system functionality.

In some embodiments, the LPs 102 and 104 are configured to be implantable in any chamber of the heart, namely either atrium (RA, LA) or either ventricle (RV, LV). Furthermore, for dual-chamber configurations, multiple LPs may be co-implanted (e.g., one in the RA and one in the RV, or one in the RV and one in the coronary sinus proximate the LV). Certain pacemaker parameters and functions depend on (or assume) knowledge of the chamber in which the pacemaker is implanted (and thus with which the LP is interacting; e.g., pacing and/or sensing). Some non-limiting examples include: sensing sensitivity, an evoked response algorithm, use of AF suppression in a local chamber, blanking and refractory periods, etc. Accordingly, each LP preferably knows an identity of the chamber in which the LP is implanted, and processes may be implemented to automatically identify a local chamber associated with each LP.

Also shown in FIG. 1B, the primary battery 114 has positive terminal 140 and negative terminal 142. Current from the positive terminal 140 of primary battery 114 flows through a shunt 144 to a regulator circuit 146 to create a positive voltage supply 148 suitable for powering the remaining circuitry of the pacemaker 102. The shunt 144 enables the battery current monitor 136 to provide the controller 112 with an indication of battery current drain and indirectly of device health. The illustrative power supply can be a primary battery 114.

In various embodiments, LP 102, 104 can manage power consumption to draw limited power from the battery, thereby reducing device volume. Each circuit in the system can be designed to avoid large peak currents. For example, cardiac pacing can be achieved by discharging a tank capacitor (not shown) across the pacing electrodes. Recharging of the tank capacitor is typically controlled by a charge pump circuit. In a particular embodiment, the charge pump circuit is throttled to recharge the tank capacitor at constant power from the battery.

In some embodiments, the controller 112 in one LP 102, 104 can access signals on the electrodes 108 and can examine output pulse duration from another pacemaker for usage as a signature for determining triggering information validity and, for a signature arriving within predetermined limits, activating delivery of a pacing pulse following a predetermined delay of zero or more milliseconds. The predetermined delay can be preset at manufacture, programmed via an external programmer, or determined by adaptive monitoring to facilitate recognition of the triggering signal and discriminating the triggering signal from noise. In some embodiments or in some conditions, the controller 112 can examine output pulse waveform from another leadless cardiac pacemaker for usage as a signature for determining triggering information validity and, for a signature arriving within predetermined limits, activating delivery of a pacing pulse following a predetermined delay of zero or more milliseconds.

In certain embodiments, the electrodes of an LP 102, 104 can be used to sense an intracardiac electrocardiogram (IEGM) from which atrial and/or ventricular activity can be detected, e.g., by detecting QRS complexes and/or P waves. Such an IEGM can also be used by an LP 102, 104 to time when communication pulses should be generated, since the orientation of the LPs 102, 104 relative to one another, and the distances between the LPs 102, 104, can change throughout each cardiac cycle. In other words, an LP can utilize a sensed IEGM to determine timing relative to a cardiac cycle.

FIG. 2 shows an LP 102, 104. The LP can include a hermetic housing 202 (e.g., the housing 110 in FIG. 1) with electrodes 108a and 108b disposed thereon. As shown, electrode 108a can be separated from but surrounded partially by a fixation mechanism 205, and the electrode 108b can be disposed on the housing 202. The fixation mechanism 205 can be a fixation helix, a plurality of hooks, barbs, or other attaching features configured to attach the pacemaker to tissue, such as heart tissue. The electrodes 108a and 108b are examples of the electrodes 108 shown in and discussed above with reference to FIG. 1B. One of the electrodes 108 (e.g., 108a) can function as a cathode type electrode and another one of the electrodes 108 (e.g., 108b) can function as an anode type electrode, or vice versa, when the electrodes are used for delivering stimulation. For the purpose of this discussion, the electrode 108a will often be referred to as the button electrode 108a (or the tip electrode 108a), and the electrode 108b will often be referred to as the ring electrode 108b.

The housing 202 can also include an electronics compartment 210 within the housing that contains the electronic components necessary for operation of the pacemaker, including, e.g., a pulse generator, receiver, a battery, and a processor for operation. The hermetic housing 202 can be adapted to be implanted on or in a human heart, and can be cylindrically shaped, rectangular, spherical, or any other appropriate shapes, for example.

The housing 202 can comprise a conductive, biocompatible, inert, and anodically safe material such as titanium, 316L stainless steel, or other similar materials. The housing 202 can further comprise an insulator disposed on the conductive material to separate electrodes 108a and 108b. The insulator can be an insulative coating on a portion of the housing between the electrodes, and can comprise materials such as silicone, polyurethane, parylene, or another biocompatible electrical insulator commonly used for implantable medical devices. In the embodiment of FIG. 2, a single insulator 208 is disposed along the portion of the housing between electrodes 108a and 108b. In some embodiments, the housing itself can comprise an insulator instead of a conductor, such as an alumina ceramic or other similar materials, and the electrodes can be disposed upon the housing.

As shown in FIG. 2, the pacemaker can further include a header assembly 212 to isolate the electrodes 108a and 108b. The header assembly 212 can be made from PEEK, tecothane or another biocompatible plastic, and can contain a ceramic to metal feedthrough, a glass to metal feedthrough, or other appropriate feedthrough insulator as known in the art.

The electrodes 108a and 108b can comprise pace/sense electrodes or return electrodes. A low-polarization coating can be applied to the electrodes, such as sintered platinum, platinum-iridium, iridium, iridium-oxide, titanium-nitride, carbon, or other materials commonly used to reduce polarization effects, for example. In FIG. 2, electrode 108a can be a pace/sense electrode and electrode 108b can be a return electrode. The electrode 108b can be a portion of the conductive housing 202 that does not include an insulator 208.

Several techniques and structures can be used for attaching the housing 202 to the interior or exterior wall of the heart. A helical fixation mechanism 205, can enable insertion of the device endocardially or epicardially through a guiding catheter. A torqueable catheter can be used to rotate the housing and force the fixation device into heart tissue, thus affixing the fixation device (and also the electrode 108a in FIG. 2) into contact with stimulable tissue. Electrode 108b can serve as an indifferent electrode for sensing and pacing. The fixation mechanism may be coated partially or in full for electrical insulation, and a steroid-eluting matrix may be included on or near the device to minimize fibrotic reaction, as is known in conventional pacing electrode-leads.

### Implant-to-Implant Event Messaging

LPs 102 and 104 can utilize implant-to-implant (i2i) communication through event messages to coordinate operation with one another in various manners. The terms i2i communication, i2i event messages, and i2i even markers are used interchangeably herein to refer to event related messages and IMD/IMD operation related messages transmitted from an implanted device and directed to another implanted device (although external devices, e.g., a programmer, may also receive i2i event messages). In certain embodiments, LP 102 and LP 104 operate as two independent leadless pacers maintaining beat-to-beat dual-chamber functionality via a “Master/Slave” operational configuration. For descriptive purposes, the ventricular LP 104 shall often be referred to as “vLP” and the atrial LP 102 shall often be referred to as “aLP”. LP 102, 104 that is designated as the master device (e.g. vLP) may implement all or most dual-chamber diagnostic and therapy determination algorithms. For purposes of the following illustration, it is assumed that the vLP is a “master” device, while the aLP is a “slave” device. Alternatively, the aLP may be designated as the master device, while the vLP may be designated as the slave device. The master device orchestrates most or all decision-making and timing determinations (including, for example, rate-response changes).

In accordance with certain embodiments, methods are provided for coordinating operation between first and second LPs configured to be implanted entirely within first and second chambers of the heart. It is alternatively possible that an LP is implanted on an exterior of a cardiac chamber, rather than within a cardiac chamber. In certain embodiments, a method transmits an event marker using conducted communication through electrodes located along a housing of the first LP, wherein the event marker is indicative of one of a local paced or sensed event. The method detects, over a sensing channel, the event marker at the second LP. The method identifies the event marker at the second LP based on a predetermined pattern configured to indicate that an event of interest has occurred in a remote chamber. In response to the identifying operation, the method initiates a related action in the second LP.

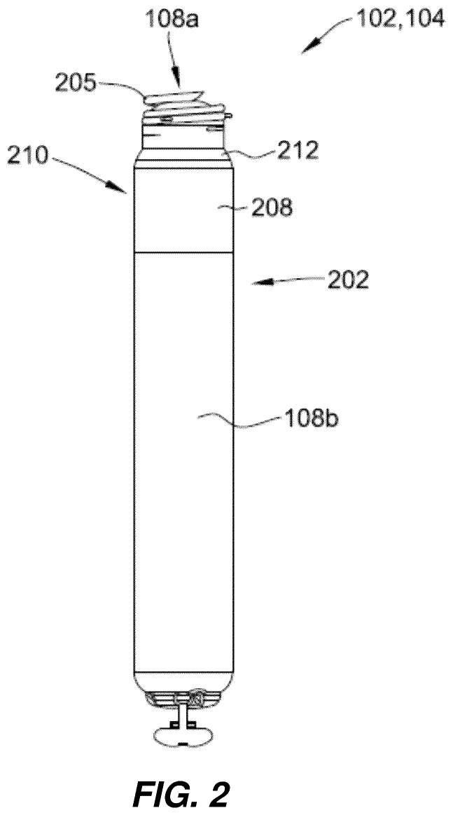

FIG. 3 is a timing diagram 300 demonstrating one example of a conducted i2i communication for a paced event. The i2i communication may be transmitted, for example, from LP 102 to LP 104. As shown in FIG. 3, in this embodiment, an i2i transmission 302 is sent prior to delivery of a pace pulse 304 by the transmitting LP (e.g., LP 102). This enables the receiving LP (e.g., LP 104) to prepare for the remote delivery of the pace pulse. The i2i transmission 302 includes an envelope 306 that may include one or more individual pulses. For example, in this embodiment, envelope 306 includes a low frequency pulse 308 followed by a high frequency pulse train 310. Low frequency pulse 308 lasts for a period T_{i2iLF}, and high frequency pulse train 310 lasts for a period T_{i2iHF}. The end of low frequency pulse 308 and the beginning of high frequency pulse train 310 are separated by a gap period, T_{i2iGap}.

As shown in FIG. 3, the i2i transmission 302 lasts for a period T_{i2iP}, and pace pulse 304 lasts for a period Tpace. The end of i2i transmission 302 and the beginning of pace pulse 304 are separated by a delay period, T_{delayp}. The delay period may be, for example, between approximately 0.0 and 10.0 milliseconds (msec), particularly between approximately 0.1 msec and 2.0 msec, and more particularly approximately 1.0 msec. The terms approximately and about, as used herein, mean +/−10% of a specified value.

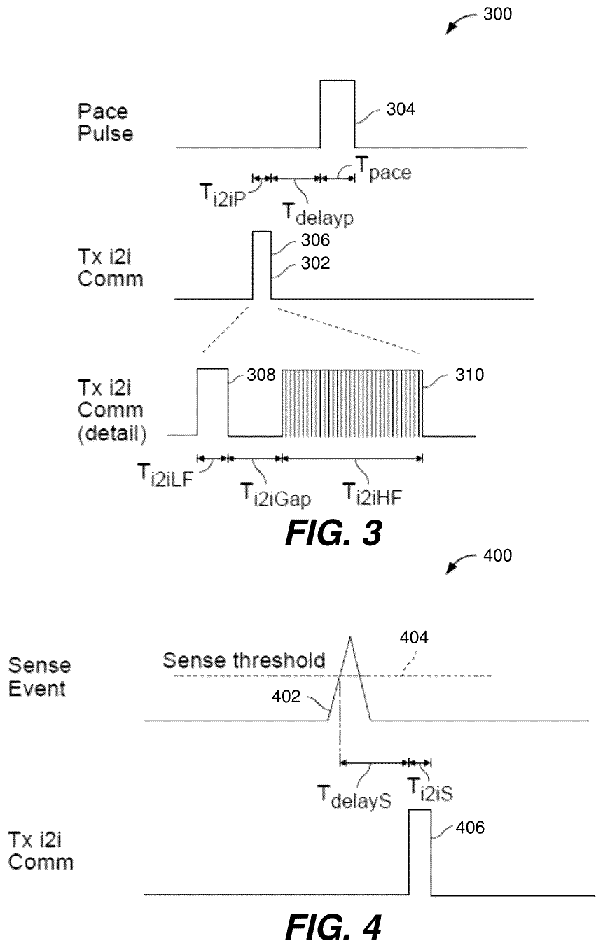

FIG. 4 is a timing diagram 400 demonstrating one example of an i2i communication for a sensed event. The i2i communication may be transmitted, for example, from LP 102 to LP 104. As shown in FIG. 4, in this embodiment, the transmitting LP (e.g., LP 102) detects the sensed event when a sensed intrinsic activation 402 crosses a sense threshold 404. A predetermined delay period, T_{delayS}, after the detection, the transmitting LP transmits an i2i transmission 406 that lasts a predetermined period T_{i2iS}. The delay period may be, for example, between approximately 0.0 and 10.0 milliseconds (msec), particularly between approximately 0.1 msec and 2.0 msec, and more particularly approximately 1.0 msec.

As with i2i transmission 302, i2i transmission 406 may include an envelope that may include one or more individual pulses. For example, similar to envelope 306, the envelope of i2i transmission 406 may include a low frequency pulse followed by a high frequency pulse train.

In accordance with certain embodiments, the duration of each of the periods T_{i2iP} and T_{i2iS} can be in the range of 1.5 msec to 13 msec, and more preferably, be within the range of 2.0 msec to 5.0 msec. This duration includes the both the low frequency pulse (e.g., 308) and the high frequency pulse train (e.g., 310, that follows the low frequency pulse). The low frequency pulse (e.g., 308) can be used as a wakeup pulse, and the high frequency pulse train (e.g., 310, that follows the low frequency pulse) can be used as a communication event marker and payload.

Where i2i communication is performed using conducted communication, it will often be referred to herein more specifically as conducted i2 communication, or equivalently i2i conducted communication. It is also noted that the phase conducted communication is often used interchangeably with the phase conductive communication.

Optionally, wherein the first LP is located in an atrium and the second LP is located in a ventricle, the first LP produces an AS/AP event marker to indicate that an atrial sensed (AS) event or atrial paced (AP) event has occurred or will occur in the immediate future. For example, the AS and AP event markers may be transmitted following the corresponding AS or AP event. Alternatively, the first LP may transmit the AP event marker slightly prior to delivering an atrial pacing pulse. Where the first LP is located in an atrium and the second LP is located in a ventricle, the second LP can initiate an atrioventricular (AV) interval after receiving an AS or AP event marker from the first LP; and can initiate a post atrial ventricular blanking (PAVB) interval after receiving an AP event marker from the first LP.

Optionally, the first and second LPs may operate in a “pure” master/slave relation, where the master LP delivers “command” markers in addition to or in place of “event” markers. A command marker directs the slave LP to perform an action such as to deliver a pacing pulse and the like. For example, when a slave LP is located in an atrium and a master LP is located in a ventricle, in a pure master/slave relation, the slave LP delivers an immediate pacing pulse to the atrium when receiving an AP command marker from the master LP.

In accordance with some embodiments, communication and synchronization between the aLP and vLP is implemented via conducted communication of markers/commands in the event messages (per an i2i communication protocol). In accordance with certain embodiments, conducted communication represents event messages transmitted from the sensing/pacing electrodes at frequencies outside the RF or Wi-Fi frequency range. Such conducted communication relies on electrical communication signals or pulses that “conducted” through a patient's body, and more specifically, through patient tissue. The figures and corresponding description below illustrate non-limiting examples of markers that may be transmitted in event messages. The figures and corresponding description below also include the description of the markers and examples of results that occur in the LP that receives the event message. Table 1 represents exemplary event markers sent from the aLP to the vLP, while Table 2 represents exemplary event markers sent from the vLP to the aLP. In the master/slave configuration, AS event markers are sent from the aLP each time that an atrial event is sensed outside of the post ventricular atrial blanking (PVAB) interval or some other alternatively-defined atrial blanking period. The AP event markers are sent from the aLP each time that the aLP delivers a pacing pulse in the atrium. The aLP may restrict transmission of AS markers, whereby the aLP transmits AS event markers when atrial events are sensed both outside of the PVAB interval and outside the post ventricular atrial refractory period (PVARP) or some other alternatively-defined atrial refractory period. Alternatively, the aLP may not restrict transmission of AS event markers based on the PVARP, but instead transmit the AS event marker every time an atrial event is sensed.

TABLE 1
“A2V” Markers / Commands (i.e., from aLP to vLP)

| Marker | Description | Result in vLP |
|---|---|---|
| AS | Notification of a sensed event | Initiate AV interval (if not in |
| | in atrium (if not in PVAB or | PVAB or PVARP) |
| | PVARP) | |
| AP | Notification of a paced event in | Initiate PAVB |
| | atrium | Initiate AV interval (if not in |
| | | PVARP) |

As shown in Table 1, when an aLP transmits an event message that includes an AS event marker (indicating that the aLP sensed an intrinsic atrial event), the vLP initiates an AV interval timer. If the aLP transmits an AS event marker for all sensed events, then the vLP would preferably first determine that a PVAB or PVARP interval is not active before initiating an AV interval timer. If however the aLP transmits an AS event marker only when an intrinsic signal is sensed outside of a PVAB or PVARP interval, then the vLP could initiate the AV interval timer upon receiving an AS event marker without first checking the PVAB or PVARP status. When the aLP transmits an AP event marker (indicating that the aLP delivered or is about to deliver a pace pulse to the atrium), the vLP initiates a PVAB timer and an AV interval time, provided that a PVARP interval is not active. The vLP may also blank its sense amplifiers to prevent possible crosstalk sensing of the remote pace pulse delivered by the aLP.

TABLE 2
“V2A” Markers / Commands (i.e., from vLP to aLP)

| Marker | Description | Result in aLP |
|---|---|---|
| VS | Notification of a sensed event | Initiate PVARP |
| | in ventricle | |
| VP | Notification of a paced event in | Initiate PVAB |
| | ventricle | Initiate PVARP |
| AP | Command to deliver | Deliver immediate pace |
| | immediate pace pulse in | pulse to atrium |
| | atrium | |

As shown in Table 2, when the vLP senses a ventricular event, the vLP transmits an event message including a VS event marker, in response to which the aLP may initiate a PVARP interval timer. When the vLP delivers or is about to deliver a pace pulse in the ventricle, the vLP transmits VP event marker. When the aLP receives the VP event marker, the aLP initiates the PVAB interval timer and also the PVARP interval timer. The aLP may also blank its sense amplifiers to prevent possible crosstalk sensing of the remote pace pulse delivered by the vLP. The vLP may also transmit an event message containing an AP command marker to command the aLP to deliver an immediate pacing pulse in the atrium upon receipt of the command without delay.

The foregoing event markers are examples of a subset of markers that may be used to enable the aLP and vLP to maintain full dual chamber functionality. In one embodiment, the vLP may perform all dual-chamber algorithms, while the aLP may perform atrial-based hardware-related functions, such as PVAB, implemented locally within the aLP. In this embodiment, the aLP is effectively treated as a remote ‘wireless’ atrial pace/sense electrode. In another embodiment, the vLP may perform most but not all dual-chamber algorithms, while the aLP may perform a subset of diagnostic and therapeutic algorithms. In an alternative embodiment, vLP and aLP may equally perform diagnostic and therapeutic algorithms. In certain embodiments, decision responsibilities may be partitioned separately to one of the aLP or vLP. In other embodiments, decision responsibilities may involve joint inputs and responsibilities.

In an embodiment, ventricular-based pace and sense functionalities are not dependent on any i2i communication, in order to provide safer therapy. For example, in the event that LP to LP (i2i) communication is lost (prolonged or transient), the system 100 may automatically revert to safe ventricular-based pace/sense functionalities as the vLP device is running all of the necessary algorithms to independently achieve these functionalities. For example, the vLP may revert to a VVI mode as the vLP does not depend on i2i communication to perform ventricular pace/sense activities. Once i2i communication is restored, the system 100 can automatically resume dual-chamber functionalities.

In certain embodiments, the LP (or other type of IMD) that receives any i2i communication from another LP (or another type of IMD) or from an external device may transmit a receive acknowledgement (ACK) indicating that the receiving LP/IMD received the i2i communication, etc. In certain embodiments, a first LP (or other type of IMD) that transmits an i2i communication to a second LP (or other type of IMD) can determine that the i2i communication failed if the first LP (or other type of IMD) does not receive an ACK from the second LP (or other type of IMD) to which the i2i communication was sent within a specified window following the transmission of the i2i communication, or in a next i2i communication received from the second LP (or other type of IMD).

As explained herein, communication and synchronization between an aLP and vLP is implemented via conducted communication of event markers (per an i2i communication protocol). In certain embodiments, the i2i communication markers may be emitted only substantially concurrent with a local pace or sense event. As such, there is no risk of emitting a marker during a vulnerable period, and thus no risk of inducing unintended excitations. The i2i communication event markers are optionally expanded with a code to indicate whether the transmitting device successfully received a valid i2i marker from the remote LP since the last transmission from the remote LP. For example, a simple example of this coding uses a binary indicator (e.g., 0/1, ACK/nACK, etc.). Optionally, more sophisticated coding schemes could alternatively be employed to include expanded information, e.g., number of consecutive missed markers, handshaking to provide insight into which marker(s) were missed, etc. These “acknowledgement codes” may be used by both LPs to diagnosis bidirectional and/or unidirectional breakdowns in i2i communication, so that the LP can take remedial actions as appropriate. One such remedial action (e.g., for transient losses of i2i communication) would be to “bridge” one or more missed/corrupted markers for n future cycles before reverting to an “i2i safe” operating mode. Another such remedial action (e.g., for more prolonged losses of i2i communication) would be to revert to an “i2i safe” mode so as to avoid possibilities of asynchronous pacing by aLP and vLP.

One example of an “i2i safe” mode is the transition from dual-chamber functionality to ventricular-only functionality (e.g., DDDR to VVIR). The LPs would exit from “i2i safe” mode and return to the programmed dual-chamber mode once bidirectional i2i communication has been reestablished. As another example of an alternative “i2i safe” mode, when the LPs experience unidirectional loss of V2A i2i communication (i.e., A2V i2i communication remains intact), the aLP and vLP may transition from DDDR to VDDR. Certain embodiments of the present technology described below increase the probability that conducted i2i communication between LPs can be maintained, and thereby reduces the probability that an “i2i safe” mode needs to be used.

As noted above, when using a pair of LPs (e.g., 102, 104) to perform pacing and/or sensing operations in the RA and RV, one of the challenges is that i2i communication is relied upon to maintain appropriate synchrony between the RV and the RA. As also noted above, a transmitter (e.g., 118) of an LP 102, 104 may be configured to transmit event messages in a manner that does not inadvertently capture the heart in the chamber where LP 102, 104 is located, such as when the associated chamber is in a refractory state. In addition, an LP 102, 104 that receives an event message may enter an “event refractory” state (or event blanking state) following receipt of the event message. The event refractory/blanking state may be set to extend for a determined period of time after receipt of an event message in order to avoid the receiving LP 102, 104 from inadvertently sensing another signal as an event message that might otherwise cause retriggering. For example, the receiving LP 102, 104 may detect a conducted communication pulse from another LP 102, 104. The amplitude of a detected (i.e., sensed) conducted communication pulse can be referred to as the sensed amplitude.

As noted above, i2i conducted communication can be adversely affected by the orientation of the LPs relative to one another and the distance between the LPs. This can also be the case where an LP communicates with another type of IMD, besides another LP. Both computer simulations and animal testing have showed that sensed amplitude varies widely with different orientation angles between LPs (or between an LP and another IMD). For example, where a first LP (e.g., 102) transmits a pulse having a pulse amplitude of 2.5V to a second LP (e.g., 104), the sensed amplitude of the pulse received by the second LP (e.g., 104) could vary from about 2 mV to less than 0.5 mV, depending upon the orientation between the first and second LPs (e.g., 102 and 104). For example, where the LP 102 is implanted in the right atrium (RA), and the LP 104 is implanted in the left atrium (LA), e.g., as shown in FIG. 1A, the distance between and the orientation of the LPs 102 and 104 relative to one another can change over the course of each cardiac cycle. Additionally, the orientation and distance of the LPs 102 and 104 relative to one another can be affected by the posture of the patient. Accordingly, since the sensed amplitude of a pulse received by one LP (e.g., 104) from the other LP (e.g., 102) can significantly vary based on the distance between and the orientation of the LPs relative to one another, the sense amplitude can significantly vary depending upon the timing of when a pulse (a conducted communication pulse) is transmitted during a cardiac cycle, as well as the posture of the patient when the pulse is transmitted.

Assume, for example, that an LP 102, 104 has a 0.5 mV sense threshold, meaning that a sensed pulse must have an amplitude of at least 0.5 mV in order to be detected as a communication pulse by the receiving LP. In other words, if sensed amplitudes of received communication pulses are below the sense threshold, the receiving LP will fail to receive the information encoded therein and may fail to respond accordingly, which is undesirable.

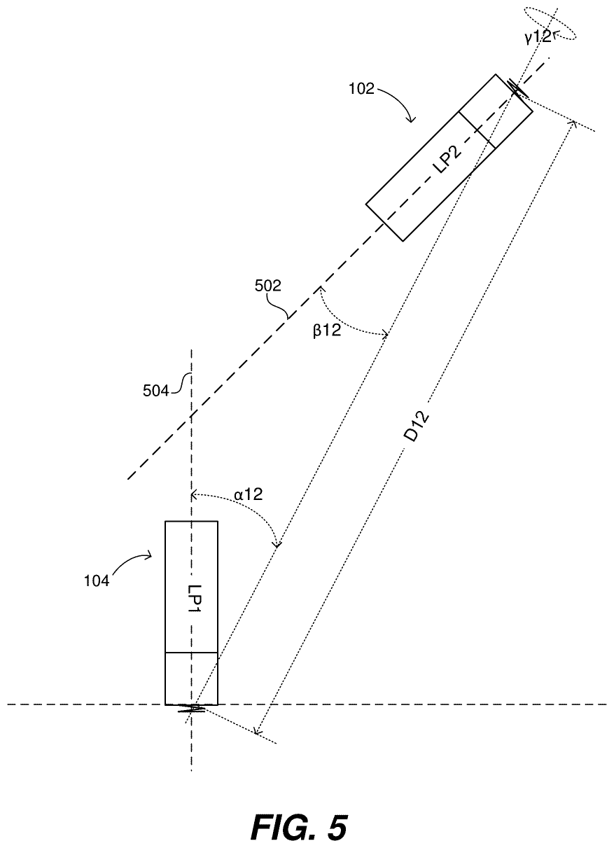

FIG. 5 is a diagram that is used to show how the orientation of two different LPs (e.g., 102, 104), labeled LP2 and LP1 in FIG. 5, can be quantified. Referring to FIG. 5, the LP2 (e.g., 102) is shown as having an axis 502, and the LP1 (e.g., 104) is shown as having an axis 504. The line D12 represents the distance between the tip (aka button) electrodes of the LP1 and the LP2. In FIG. 5, the angle α12 is the angle between the axis 504 of the LP1 and the line D12; the angle β12 is the angle between the axis 502 of the LP2 and the line D12; and the angle γ12 is angle between the plane defined by the angle α12 and the plane defined by the angle β12.

Table 3, below, provides the results of simulations that show how sensed amplitudes are affected by the orientation of LP1 and LP2 relative to one another, where the LP2 is assumed to be implanted in the RA, the LP1 is assumed to be implanted in the RV, and the distance D12 is assumed to be fixed at 124 millimeters (mm). In Table 3, it is assumed that the distance D12 between the LP1 and the LP2 remains constant, so that just the orientation of LP1 and LP2 relative to one another can be analyzed. Since the distance D12 is the distance between the button (aka tip) electrodes of the LP1 and the LP2, the distance D12 can also be referred to herein as the LP1button-LP2button distance.

TABLE 3

| Distance | Angle | Angle | | |
|---|---|---|---|---|
| D12 | α12 | β12 | RA → RV | RA ← RV |
| 124 mm | 20° | 12° | 2.5 V → | 2.11 mV |
| | | | 2.13 mV | ←2.5 V |
| 124 mm | 20° | 32° | 2.5 V → | N/A |
| | | | 1.82 mV | |
| 124 mm | 20° | 52° | 2.5 V → | N/A |
| | | | 1.32 mV | |
| 124 mm | 20° | 72° | 2.5 V → | N/A |
| | | | 0.745 mV | |
| 124 mm | 20° | 82° | 2.5 V → | 0.460 mV |
| | | | 0.470 mV | ←2.5 V |
| 124 mm | 20° | 92° | 2.5 V → | 0.198 mV |
| | | | 0.198 mV | ←2.5 V |
| 124 mm | 10° | 82° | 2.5 V → | N/A |
| | | | 0.6627 mV | |
| 124 mm | 40° | 82° | 2.5 V | N/A |
| | | | → −0.1135 mV | |
| 124 mm | 50° | 82° | N/A | N/A |

The first row of Table 3 shows that when the angle β12 (i.e., the angle between the axis 502 of the LP2 and the line D12) is 12 degrees, in response to the LP2 transmitting a communication pulse having an amplitude of 2.5V, the sense amplitude of the communication pulse received by the LP1 will be 2.13 mV, which is well above a 0.5 mV sense threshold. By contrast, the sixth row of Table 3 shows that when the angle β12 is 92 degrees, in response to the LP2 transmitting a communication pulse having an amplitude of 2.5V, the sense amplitude of the communication pulse received by the LP1 will be only 0.198 mV, which is well below the 0.5 mV sense threshold. Looking at the right most column and the first row of Table 3 shows that when the angle β12 is 12 degrees, in response to the LP1 transmitting a communication pulse having an amplitude of 2.5V, the sense amplitude of the communication pulse received by the LP2 will be 2.11 mV, which is well above a 0.5 mV sense threshold; and when the angle β12 is 92 degrees, in response to the LP1 transmitting a communication pulse having an amplitude of 2.5V, the sense amplitude of the communication pulse received by the LP2 will be only 0.198 mV, which is well below the 0.5 mV sense threshold.

With larger heart sizes, the sensed amplitudes decrease. More specifically, a larger heart can cause the distance D12 between the LP1 and the LP2 to increase, with the results summarized in Table 4, below.

TABLE 4

| Distance | Angle | Angle | | |
|---|---|---|---|---|
| D12 | α12 | β12 | RA → RV | RA ← RV |
| 150 mm | 20° | 12° | 2.5 V → 0.96 mV | N/A |
| 150 mm | 20° | 32° | 2.5 V → 0.76 mV | N/A |
| 150 mm | 20° | 52° | 2.5 V → 0.51 mV | N/A |
| 150 mm | 20° | 72° | 2.5 V → 0.25 mV | N/A |
| 150 mm | 20° | 82° | 2.5 V → 0.12 mV | N/A |
| 150 mm | 20° | 92° | 2.5 V → 0.005 mV | N/A |
| 150 mm | 20° | 52° | 2.5 V → 0.51 mV | N/A |
| 150 mm | 10° | 52° | 2.5 V → 0.59 mV | N/A |
| 150 mm | 40° | 52° | 2.5 V → 0.27 mV | N/A |

The results summarized in Table 4 mimic a worst case where the heart size is at the upper bounds (D12˜150 mm). As can be appreciated from a comparison between Table 4 and Table 3, the sensed amplitudes decreased as D12 was increased from 124 mm to 150 mm, so that in Table 4 when the angle β12 is greater than 52 degree, the sensed amplitude is lower than the 0.5 mV sense threshold. Accordingly, it can be appreciated that conducted i2i communication between LPs implanted in larger hearts are even more adversely affected than smaller hearts by the relative orientation of the LPs. It can be appreciated from Table 4 that that i2i communication can be adversely affected by the distance between LPs.

When performing i2i communication, the one or more pulses that are transmitted from one LP to another LP can be referred more generally as the i2i signal. Due to the nature of electrode potential distribution, bipolar sensing of the i2i signal (by the LP that is receiving/sensing the i2i signal) is minimal along iso-potential lines and maximum along lines orthogonal to the iso-potential lines. In other words, when the respective axes (e.g., 502 and 504 in FIG. 5) of the two LPs (communicating with one another) are aligned with one another the sensed i2i signal is near its maximum, and when the respective axes (e.g., 502 and 504 in FIG. 5) of the two LPs are orthogonal to one another the sensed i2i signal is near its minimum.

For the purpose of this discussion, when LPs are oriented relative to another such that (for a give transmitted communication pulse amplitude) the sense amplitude of the communication pulse received by an LP will be below the sense threshold (e.g., 0.5 mV), the LPs can be said to be within a “deaf zone”. This is because under such circumstances the LPs cannot successfully communicate or “hear” one another even though they are attempting to communicate or “talk” with one another.

For the purpose of this discussion, it is assumed that an LP system includes a vLP implanted in or on a ventricular chamber (e.g., the right ventricular chamber) and an aLP implanted on or an atrial chamber (e.g., the right atrial chamber). In such an LP system, the relative locations and orientations of the vLP and aLP varies over a cardiac cycle. When heart fills with blood, the heart is at its maximum volume and the vLP and the aLP are typically relatively far apart. By contrast, when the heart contracts, the vLP and the aLP come closer to one another and their orientation relative to one another changes.

Certain embodiments of the present technology described herein leverage the fact that when a heart (in and/or on which LPs are implanted) contracts, a distance is typically reduced between the LPs, and an orientation between the LPs changes. The distance between LPs and the orientation of the LPs relative to one another are the two parameters that affect the success of conducted i2i communication.

### Improved Conducted i2i Communication

In accordance with certain embodiments of the present technology, an aLP (e.g., implanted in or on the right atrial chamber) normally uses a primary conducted communication protocol (PCCP) to communicate with a vLP (e.g., implanted in or on the right ventricular chamber) during a primary communication window (PCW), wherein the PCW is a temporal window that is in close proximity to paced and sensed atrial events. More specifically, when using the PCCP, the aLP transmits an i2i conducted communication message to the vLP immediately before pacing the atrial chamber to cause an atrial paced event, and the aLP transmits an i2i conducted communication message to the vLP immediately after sensing an atrial sensed event. Alternatively, when using the PCCP, the aLP transmits an i2i conducted communication message to the vLP immediately after pacing the atrial chamber to cause an atrial paced event. The term “immediately before” as used herein means within 50 milliseconds (msec) before, preferably within 20 msec before, and more preferably within 5 msec before. Similarly, the term “immediately after” as used herein means within 50 msec after, preferably within 20 msec after, and more preferably within 5 msec after. The vLP can then, in response to successfully receiving the i2i conducted communication message from the aLP, start an AV interval timer that the vLP uses to determine when the vLP should pace the ventricular chamber at the AV delay following the atrial paced or sensed event. In view of the above discussion, it can be appreciated that the PCW is a window that is within 50 msec of a paced or sensed event, is more preferably within 20 msec of a paced or sensed event, and is even more preferably within 5 msec of a paced or sensed event.

In certain embodiments, the vLP, in response to successfully receiving the i2i conducted communication message from the aLP, transmits an i2i conducted communication message to the aLP that acknowledges that the vLP successfully received the message from the aLP. Such a message can be referred to as an acknowledgement (ACK) message. By contrast, if the vLP does not successfully receiving an i2i conducted communication message from the aLP within a specified window of time, e.g., within the PCW, the vLP can transmit an i2i conducted communication message to the aLP that informs the aLP that the vLP failed to receive a message from the aLP within the PCW, wherein such a message can be referred to herein as a negative ACK message, or an nACK message. It is also possible that the ACK and nACK messages be included in a next message sent by a vLP, e.g., as part of a header of payload of an event message.

Alternatively, if the vLP does not successfully receive an i2i conducted communication message from the aLP within the PCW, the vLP may simply not transmit an ACK message to the aLP, and the aLP can be configured to interpret not receiving an ACK message as an indication that the vLP did not receive message that originated from the aLP.

Similarly, when a vLP (e.g., implanted in or on the right ventricular chamber) uses the PCCP to communicate with an aLP (e.g., implanted in or on the right atrial chamber), the vLP can transmit an i2i conducted communication message to the aLP immediately before (or immediately after) pacing the ventricular chamber to cause a ventricular paced event, or the vLP can transmit an i2i conducted communication message to the aLP immediately after sensing a ventricular sensed event. The aLP can then, in response to successfully receiving the i2i conducted communication message from the vLP, start an VA interval timer that the aLP uses to determine when the aLP should pace the atrial chamber at the VA delay following the ventricular paced or sensed event, in the absence of an intrinsic atrial event occurring during the VA interval.

The aLP can additionally, in response to successfully receiving the i2i conducted communication message from the vLP, transmit an i2i conducted communication message to the vLP that acknowledges that the aLP successfully received the message from the vLP, wherein such a message can be referred to as an ACK message. By contrast, if the aLP does not successfully receive an i2i conducted communication message from the vLP within a specified window of time, e.g., within a PCW, the aLP can transmit an i2i conducted communication message to the vLP that informs the vLP that the aLP failed to receive a message from the vLP within the PCW, wherein such a message can be referred to herein as a negative ACK message, or an nACK message. It is also possible that the ACK and nACK messages be included in a next message sent by the aLP, e.g., as part of a header of payload of an event message.

Alternatively, if the aLP does not successfully receive an i2i conducted communication message from the vLP within the PCW, the aLP may simply not transmit an ACK message to the vLP, and the vLP can be configured to interpret not receiving an ACK message as an indication that the aLP did not receive message that originated from the vLP.

In accordance with certain embodiments of the present technology, the aLP and/or the vLP can monitor a quality metric of the conducted i2i communication, while the aLP and the vLP are performing the conducted communication with one another in accordance with the PCCP. Then, if the quality metric falls below a specified threshold for a specified amount of time or a specified number of cardiac cycles, which is indicative of poor i2i conducted communication, there can be a transition from transmitting i2i conducted communication messages within the PCW in accordance with the PCCP, to transmitting i2i conducted communication messages within an alternative communication window (ACW) in accordance with an alternative conducted communication protocol (ACCP). As will be discussed in additional detail below, the quality metric can be indicative of communication throughput, but is not limited thereto. The specified amount of time can be, e.g., 1, 2, or 5 seconds, but is not limited thereto. The specified number of cardiac cycles can be, e.g., 1, 2 or 5 cardiac cycles, but is not limited thereto.

In accordance with certain embodiments, the only difference between the ACCP and the PCCP is that the ACCP uses the ACW for performing conducted i2i communication, in contrast to the PCCP using the PCW for performing conducted i2i communication. In other embodiments, there can also be additional difference between the ACCP and the PCCP, e.g., conducted communication pulses can have a different (e.g., higher or lower) amplitude when the ACCP is used compared to when the PCCP is used. Additional and/or alternative variations are also possible and within the scope of the embodiments described herein.

The ACW, which is used to perform i2i conducted communication in accordance with the ACCP, can be determined in various different manners. In certain embodiments, the ACW is identified for use with a specific patient while the patient is visiting a clinician, immediately after LP(s) and/or other type(s) of IMD(s) have been implanted in a patient, and/or at a later time when the patient is visiting the clinician. The ACW can be identified, for example, by testing the transmission and reception of i2i conducted communication messages between the LPs at various different windows (temporal windows) that are timed relative to paced and sensed events, as will be described in additional detail below. Once the ACW is identified, information that specifies the ACW can be stored in memory of the LPs, and the ACW can be used for performing backup i2i conducted communication, when the quality of the i2i conducted communication using the PCW drops below a specified threshold.

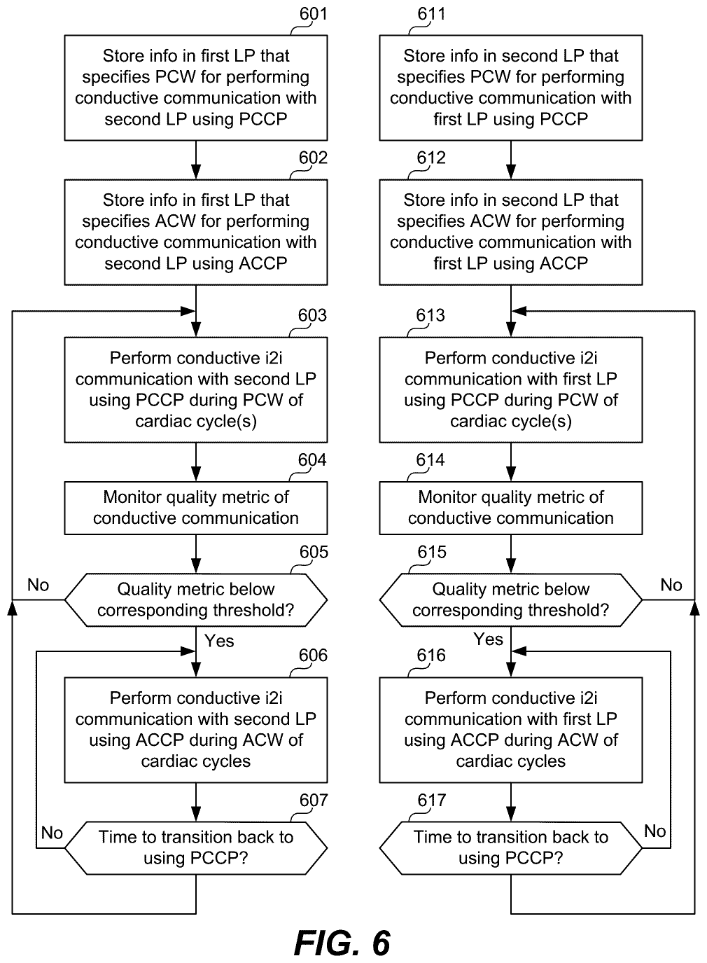

The high level flow diagram of FIG. 6 is now used to summarize certain embodiments of the present technology that were introduced above. Thereafter, additional details of various steps of the flow diagram are provided. The method summarized with reference to FIG. 6 is for use by a system including first and second LPs that are configured to be implanted, respectively, in or on first and second cardiac chambers of a patient and are configured to perform conducted i2i communication with one another to thereby enable coordinated pacing of the first and second cardiac chambers of the patient. For example, the first and second LPs can be an aLP and a vLP, wherein the aLP is implanted in or on the right atrial chamber, and the vLP is implanted in or on the right ventricle chamber. For another example, if the system is used to perform bi-ventricular pacing, then the first and second LPs can include an LP implanted in or on the right ventricular chamber, and an LP implanted in or on the left ventricular chamber. Other variations are also possible and within the scope of the embodiments described herein.

In FIG. 6, the steps shown at the left are performed by a first LP, e.g., an aLP, and the steps shown at the right are performed by a second LP, e.g., a vLP, or vice versa. For another example, the steps shown at the left can be performed by a vLP implanted in or on the right ventricular chamber, and the steps shown at the right can be performed by another vLP implanted in the left ventricular chamber, or vice versa. Other variations are also possible and within the scope of the embodiments described herein with reference to FIG. 6. For example, it is possible that the steps shown at the left can be performed by an LP and the steps performed at the right can be performed by another type of IMD (e.g., an ICD or ICM), or vice versa.

Referring to FIG. 6, step 601 involves storing information in the first LP that specifies a primary communication window (PCW) for performing conducted communication with the second LP (or other type of IMD), in accordance with a primary conducted communication protocol (PCCP). The PCW can be specified in terms of a delay relative to paced and sensed events. For example, the specified delay can be 2 msec, meaning that the PCW begins 2 msec prior to (or after) paced events, and 2 msec after sensed events. For another example, the specified delay can be 5 msec, meaning the PCW begins 5 msec prior to (or after) paced events, and 5 msec after sensed events. The specified delays discussed above, which are used with the PCCP, can also be referred to herein as a primary specified time. It is also noted that the terms “after” and “following” are often used interchangeably herein, and the terms “prior to” and “before” are often used interchangeably herein.

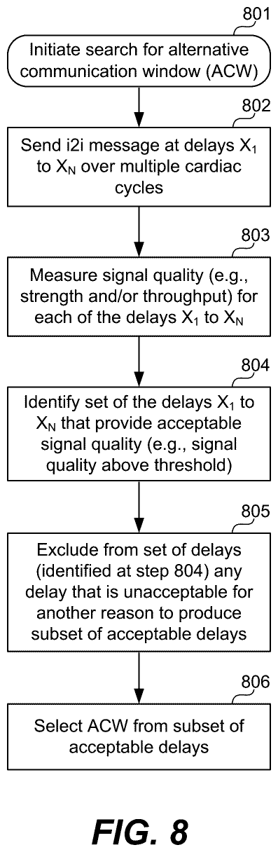

Step 602 involves storing information in the first LP that specifies an alternative communication window (ACW) for performing conducted communication with the second LP, in accordance with an alternative conducted communication protocol (ACCP). Example details of how to determine the ACW, in accordance with certain embodiments of the present technology, are described below with reference to FIG. 8.

While steps 601 and 602 are shown as separate steps in FIG. 6, they can be implemented as a single step, as two separate steps as shown, or potentially as more than two steps. Steps 601 and 602 can be performed using an external programmer (e.g., programmer 109 in FIG. 1A), or some other type of external device that is intended to wirelessly communicate with one or more IMDs, but is not limited thereto.

Step 603 involves the first LP performing conducted i2i communication with the second LP (or other type of IMD) by transmitting and/or receiving conducted communication messages to and/or from the second LP, using the PCCP during a PCW of one or more cardiac cycles. This can, for example, involve the first LP transmitting an event message to the second LP each time the first LP causes a paced event (a paced depolarization) of the first cardiac chamber, and/or each time the first LP senses an intrinsic event (an intrinsic depolarization) of the first cardiac chamber.

When using the PCCP, the first LP will transmit a paced event message to the second LP starting at a primary specified time prior to (or following) a paced event, or starting at a primary specified time following a sensed event, wherein the primary specified time prior to a paced event and the primary specified time following a sensed event may or may not be equal to one another. For the sake of this discussion, and simplicity, it is presumed that the primary specified time prior to a paced event and the primary specified time following a sensed event are the same, e.g., 5 msec. Continuing with this example, when using the PCCP, the first LP will transmit a paced event message to the second LP starting 5 msec prior to each paced event of the first cardiac chamber (i.e., prior to delivering a pacing pulse to the first cardiac chamber), and the first LP will transmit a sensed event to the second LP starting 5 msec after each sensed event of the first cardiac chamber (i.e., following sensing of an intrinsic depolarization of the first cardiac chamber). In the above example, the PCW is a window that starts 5 msec prior to a paced event, or a window that starts 5 msec after a sensed event. In accordance with certain embodiments, the first specified time should be 50 msec or less, is preferably 20 msec or less, and is more preferably 5 msec or less. Similarly, the second specified time should be 50 msec or less, is preferably 20 msec or less, and is more preferably 5 msec or less. The duration of the PCW is dependent on the duration of a pulse train of an i2i communication message that is sent during a single cardiac cycle. An example duration of the PCW is 2 msec, or 3 msec, but is not limited thereto.

Still referring to FIG. 6, at step 604 the first LP monitors a quality metric of the conducted i2i communication between the first LP and the second LP. In certain embodiments, the quality metric is indicative of the throughput of the conducted communication. Alternatively, or additionally, the quality metric can be indicative of one or more the bit error rate (BER), signal to noise ratio (SNR), maximum or average signal amplitude, a total energy of one or more conducted communication pulses received by one of the first and second LPs, and/or the like, of the conducted communication.

At step 605 the first LP determines whether the quality metric has fallen below a corresponding threshold, e.g., for at least a specified amount of time, for at least a specified number of cardiac cycles, or for at least X out of Y cardiac cycles (where Y>X, Y is at least 3, and X is at least 2), but not limited thereto. If the answer to the determination at step 605 is No, then flow returns to step 603 and the first LP continues to perform conducted i2i communication using the PCCP. If the answer to the determination at step 605 is Yes (i.e., if the quality metric has fallen below the corresponding threshold), then flow goes to step 606. At this point there is a transition from using the PCCP to using the ACCP. While steps 604 and 604 are shown as two separate steps in FIG. 6, they can be implemented as a single step.

At step 606, the first LP performs conducted i2i communication with the second LP during the ACW, in accordance with the ACCP. As noted above, example details of how to determine the ACW, in accordance with certain embodiments of the present technology, are described below with reference to FIG. 8. When using the ACCP, the first LP will transmit a paced event message to the second LP starting at an alternative specified time following a paced event or an alternative specified time following a paced event sensed, wherein the alternative specified time following to a paced event and the alternative specified time following a sensed event may or may not be equal to one another. Most likely, the alternative specified time following a paced event and the alternative specified time following a sensed event will be equal to one another, but that need not be the case.

At step 607, the first LP determines whether it is time to transition back to using the PCCP. In accordance with certain embodiments, once the first LP starts performing conducted i2i communication in accordance with the ACCP, the first LP can continue to use the ACCP for a predetermined period of time (e.g., 1 minute, 2 minutes, 5 minutes, or the like), or for a predetermined number of cardiac cycles (e.g., 60, 120, or 300 cardiac cycles, or the like). If it is not yet time to transition back to using the PCCP, then flow returns to step 606. If it is time to transition back to using the PCCP, then flow instead returns to step 603. In another embodiment, the first LP transitions back to using the PCCP when the first LP successfully receives an i2i message during the PCW. Other variations are also possible and within the scope of the embodiments described herein. In certain embodiments, once the first LP transitions from using the PCCP to using the ACCP, the first LP transmits a message during the ACW associated with the cardiac cycle, but not during the PCW associated with the cardiac cycle. In other embodiments, once the first LP transitions from using the PCCP to using the ACP, the first LP transmits a same message for a cardiac cycle during both the PCW and the ACW, and thus, sends redundant messages for the same cardiac cycle. Using both PCW and ACW to redundantly transmit a same message may be preferred in certain circumstances. For example, redundant i2i messages allow for more effective and timely transmission of critical i2i messages from one LP to another. Accordingly, in certain embodiments, whenever a critical i2i message is being sent from a first IMD to a second IMD, the critical i2i message is sent within both the PCW and the ACW, which increases the probability that the second IMD will be able to successfully receive at least one of the instances of the critical i2i message.

Steps 611, 612, 613, 614, 615, 616 and 607 are similar, respectively, to steps 601, 602, 603, 604, 605, 606 and 607, except are from the perspective of the second LP (or other type of IMD), and thus, do not need to be described in detail.

As noted above, in certain embodiments, redundant i2i messages allow for more effective and timely transmission of critical i2i messages from one LP to another. More generally, in accordance with certain embodiments, whenever a critical i2i message is sent from one IMD to another, or a critical p2i message is sent from an external device to one or more IMDs, the critical message is sent within both the PCW and the ACW, to increase the probability that the critical message will be successfully received. Critical i2i messages can include, but are not limited to, i2i messages that are intended to cause one of the following: a pacing rate change due to activity (rate responsive pacing), a mode switch upon detection atrial tachycardia, an AV/PV interval extension while searching for an intrinsic depolarization in a ventricle, or pacing hysteresis while searching from an intrinsic depolarization in atrium. An instruction to transition to magnet mode is another example of a critical message. When such critical messages are sent from one IMD to another, it is important that these messages are successfully received in a timely fashion in order to transition to a different pacing mode and/or rate without losing synchrony. Accordingly, in certain embodiments, whenever a critical i2i message is sent from on IMD to another, or a critical p2i message is sent from an external device to one or more IMDs, the critical message is sent within both the PCW and the ACW to increase the probability that the IMD (to which the critical message is sent) will be able to successfully receive at least one of the instances of the critical message.

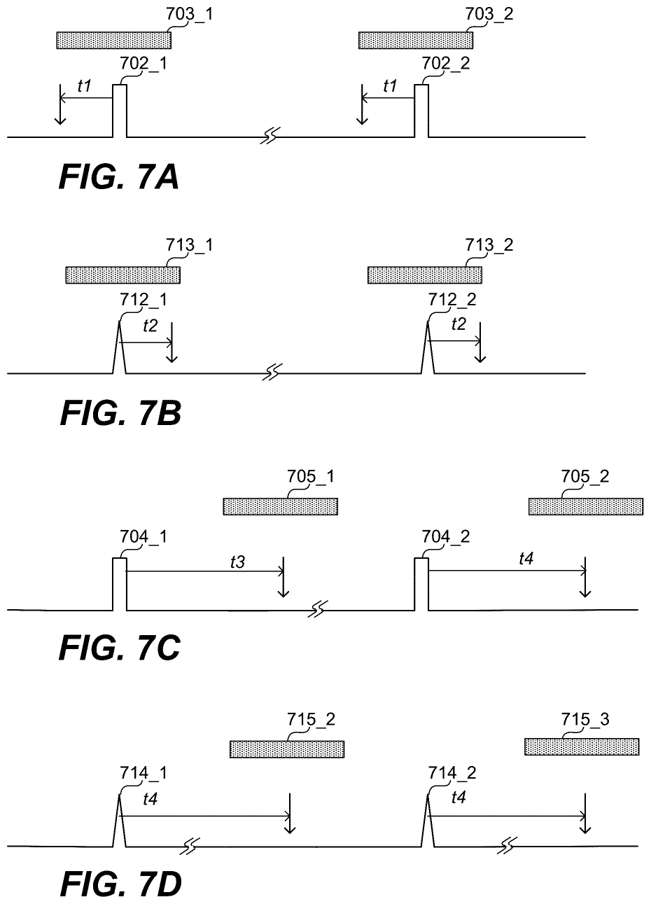

Referring to FIG. 7A, the timing diagram therein shows a first paced event 702_1 and a second paced event 702_2, and shows a respective time t1 prior to each of the paced events when an LP initiates a conducted i2i communication message that informs another LP of the paced events when utilizing the PCCP. The time between the paced events 702_1 and 702_2 is based on the present pacing rate, e.g., if the present pacing rate is 80 beats per minute (bpm), then the time between the paced events is equal to 60 seconds per minute divided by 80 bpm, which is equal to 0.75 seconds, i.e., 750 msec. Where the PCCP involves initiating a paced event message 5 msec prior to a paced event, then the time t1 in FIG. 7A is equal to 5 msec.

Referring to FIG. 7B, the timing diagram therein shows a first sensed event 712_1 and a second sensed event 712_2, and shows a respective time t2 following each of the sensed events when an LP initiates a conducted i2i communication message that informs another LP of the sensed events when utilizing the PCCP. The time between the sensed events 712_1 and 712_2 is based on the intrinsic heart rate, e.g., if the intrinsic heart rate is 80 bpm, then the time between the sensed events is equal to 750 msec. Where the PCCP involves initiating a sensed event message 5 msec after a sensed event, then the time t2 in FIG. 7B is equal to 5 msec.

Comparing FIGS. 7A and 7B to one another, it could be appreciated that in this example of the PCCP, the times within cardiac cycles at which paced event messages are initiated differ from the times within cardiac cycles at which sensed event messages are initiated, which means that the distance between and orientation of the LPs relative to one another may differ when paced event messages are sent compared to when sensed event messages are sent. However, if the times t1 and t2 are relatively small, e.g., 5 msec in the above example, that means the paced event message and the sensed event messages are sent about 10 msec apart from one another within a cardiac cycle, and thus, at almost the same times within cardiac cycles, considering the length of a cardiac cycle in this example is 750 msec. In FIG. 7A example PCWs are labeled 703_1 and 703_2, and in FIG. 7B example PCWs are labeled 713_1 and 713_2.

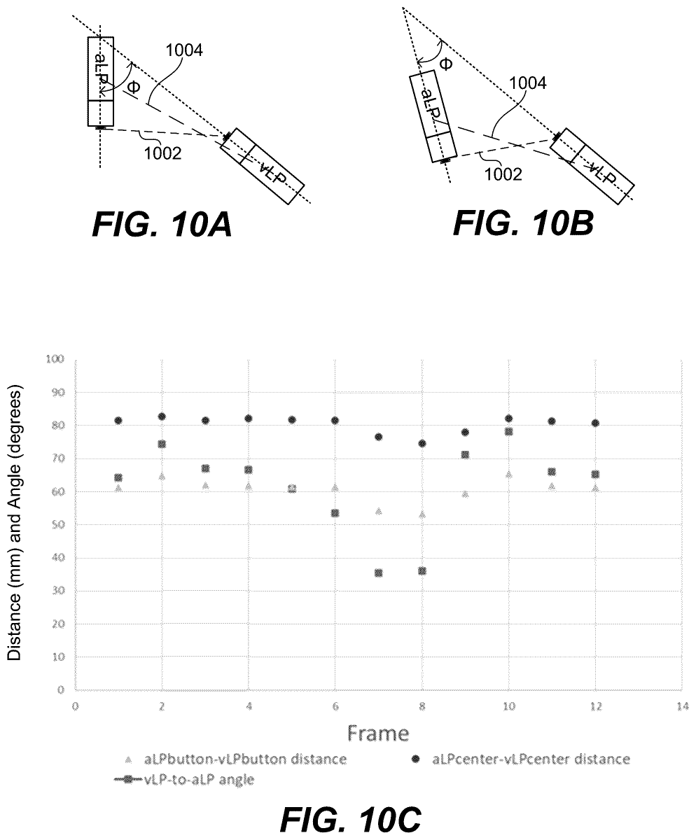

In accordance with certain embodiments, when using the ACCP, paced event messages are sent after paced events (as opposed to prior to pace events, as may also be the case when using the PCCP, depending upon implementation). This is because more favorable conditions for conducted i2i communication may occur during systole (i.e. after myocardium has been depolarized by a pace pulse). As shown in FIG. 10C, during systole a change in distance and/or relative angle between the aLP and vLP can significantly increase the communication signal amplitude. Similarly, when using the ACCP, sensed event messages are also sent after sensed events.

Referring to FIG. 7C, the timing diagram therein shows a first paced event 704_1 and a second paced event 704_2, and shows a respective time t3 after each of the paced events when an LP initiates a conducted i2i communication message that informs another LP of the paced events when utilizing the ACCP. The time between the paced events 704_1 and 704_2 is based on the present pacing rate, e.g., if the present pacing rate is 80 beats per minute (bpm), then the time between the paced events is equal to 60 seconds per minute divided by 80 bpm, which is equal to 0.75 seconds, i.e., 750 msec. Where, for example, the ACCP involves initiating a paced event message 200 msec after a paced event, then the time t3 in FIG. 7C is equal to 200 msec.

Referring to FIG. 7D, the timing diagram therein shows a first sensed event 714_1 and a second sensed event 714_2, and shows a respective time t4 following each of the sensed events when an LP initiates a conducted i2i communication message that informs another LP of the sensed events when utilizing the PCCP. The time between the sensed events 714_1 and 714_2 is based on the intrinsic heart rate, e.g., if the intrinsic heart rate is 80 bpm, then the time between the sensed events is equal to 750 msec. Where the ACCP involves initiating a sensed event message 200 msec after a sensed event, then the time t4 in FIG. 7B is equal to 200 msec.

Comparing FIGS. 7C and 7D to one another, it could be appreciated that in this example of the ACCP, the times within cardiac cycles at which paced event messages are initiated are the same as times within cardiac cycles at which sensed event messages are initiated, which means that the distance between and orientation of the LPs relative to one another will likely be very similar when paced event messages are sent compared to when sensed event messages are sent. In FIG. 7C example ACWs are labeled 705_1 and 705_2, and in FIG. 7D example ACWs are labeled 715_1 and 715_2.

The ACW is preferably selected such that it does not adversely affect a present or next paced event that is caused by the local LP sending a paced event message. Additionally, when the ACW is used, the remote LP receiving a paced (or sensed) event message should compensate for the ACW being used when the remote LP determines when it should deliver a pacing pulse, or the like. For a specific example, if an aLP transmits a paced or sensed event message within an ACW that starts 50 msec after the paced or sensed atrial event, then the vLP that receives the paced or sensed event message may reduce its programmed AV delay (aka AV interval) by 50 msec. For another example, if a vLP transmits a paced or sensed event message within an ACW that starts 50 msec after the paced or sensed ventricular event, then the aLP that receives the paced or sensed event message may reduce its programmed VA delay (aka VA interval) by 50 msec. Based on tests and simulations, it is believed that for at least some patients a preferred ACW may correspond to mechanical ventricular systole, which for at least some patients occurs about 200 msec after onset of electrical ventricular depolarization.

The high level flow diagram of FIG. 8 will now be used to describe a technique for identifying the alternative communication window (ACW) that is to be used when the alternative conducted communication protocol (ACCP) is used. Referring to FIG. 8, at step 801 a search for the ACW is initiated, e.g., by an external programmer, another type of external device, or by an IMD itself, but not limited thereto. More specifically, this could be done with the aid of a programmer in-clinic or it could also be performed as needed during remote out-of-clinic programming, or out-of-clinic automatically on a regular schedule (e.g., every specified number of hours or days, or the like).

Step 802 involves sending an i2i conducted communication message from one of the LPs to another one of the LPs at various different delays X₁ to X_{N}, so that measures of signal quality can be determined and stored at step 803 for each of the different delays X₁ to X_{N}. It is presumed that there is a handshake, and more specifically, that the LP receiving a message sends an acknowledgement (ACK) to the LP that sent the message within a specified time period (e.g., 10 msec), so that the sending LP can monitor throughput. In certain embodiments, the measure of signal quality is indicative of the throughput of the conducted communication. Alternatively, or additionally, the measure of signal quality can be indicative of one or more the bit error rate (BER), signal to noise ratio (SNR), maximum or average signal amplitude, a total energy of one or more conducted communication pulses received by one of the first and second LPs, and/or the like, of the conducted communication. It is possible that multiple i2i communication messages are sent at each of the delays and the measures of signal quality determined for the same delay are combined (e.g., added or averaged) to provide for more accurate measures. In certain embodiments, the i2i communication messages that are sent at different delays, for the purpose of identifying the ACW, can be similar to the normal i2i communication messages that are transmitted within the PCW, but with a special encoding differentiating them from a sense or pace marker preamble.

Still referring to FIG. 8, step 804 involves identifying which delays provide acceptable signal quality, e.g., which can be determined by comparing the measure(s) of signal quality to one or more appropriate threshold(s). The result of step 804 is a set of delays that provide acceptable signal quality. There may be certain delays that should not be used. More specifically, it may be that delays that are within refractory periods are preferred over delays that are outside refractory periods, to reduce the chance that conducted i2i communication messages may inadvertently cause capture. Step 805 involves excluding from the set of delays, which had been determined at step 804, those delays that should not be used, and more generally, that is/are unacceptable for one or more predetermined reasons.

Step 806 involves selecting one of the delays to use for the beginning of the ACW. In certain embodiments, step 806 can involve selecting the ACW that provides for the best measure of signal quality.

In certain embodiments, the duration of the ACW is predetermined, and what is really being determined using the technique described with reference to FIG. 8 is the timing of the ACW relative to paced and sensed events.

In certain embodiments, the steps summarized with reference to FIG. 8 can be performed for each of a plurality of different postures, and a different respective ACW can be determined and stored for each of the plurality of different postures. In such embodiments, whenever LPs (or an LP and another type of IMD) are to utilize an ACW for performing conducted i2i communication, at least one of the LPs (or the LP and other type of IMD) determines the posture of the patient (e.g., based on one or more outputs of an accelerometer) and select the appropriate ACW to use for the posture.

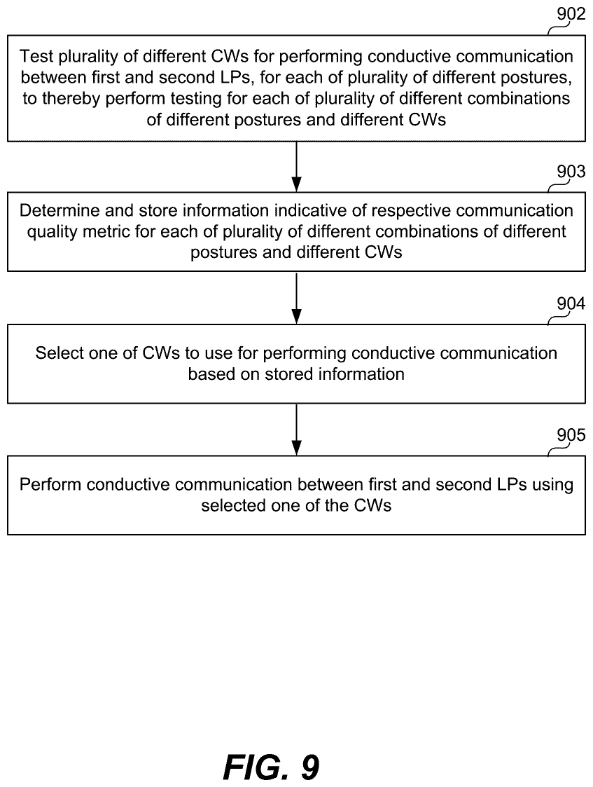

The ACW is an example of a communication window (CW). The measure of quality associated with an ACW, and more generally a CW, can be dependent on the specific posture of the patient, as the relative distance and orientation of LPs to one another throughout a cardiac cycle may vary depending upon whether a patient is standing, sitting, lying one their right side, lying on their left side, etc. Accordingly, referring to FIG. 9, in certain embodiments at step 902 a plurality of different communication windows (CWs) for performing conducted i2i communication between first and second LPs can be tested, for each of a plurality of different postures of the patient, to thereby perform the testing for each of a plurality of different combinations of the different postures and the different CWs. In certain embodiments, each of the plurality of CWs corresponds to a different delay relative to a paced or sensed event. Step 903 involves determining and storing information indicative of a respective communication quality metric for each of the plurality of different combinations of the different postures and the different CWs. Step 904 involves selecting one of the CWs to use for performing the conducted communication based on the stored information. In certain embodiments, step 904 is performed by selecting one of the CWs that, based on the stored information, should result in the conducted communication being successful regardless of which one of the plurality of different postures the patient is positioned. Step 905 involves performing the conducted communication between the first and second LPs using the selected one of the CWs.

As was explained above, the relative distance between LPs, and the orientation of LPs relative to one another can vary throughout a cardiac cycle. This is even the case when the patient remains in a same posture. Further, how the distance and orientation changes throughout a cardiac cycle will also depend on the posture of the patient. For example, changes in the distance and orientation between an aLP and a vLP over a cardiac cycle while a patient is standing will likely differ from the changes in the distance and orientation between the aLP and the vLP over a cardiac cycle while the patient is lying one their right side, or lying on their left side, etc.

FIGS. 10A and 10B are examples of the distance and orientation between an aLP and a vLP at multiple different times during a cardiac cycle, while the patient is in the same orientation (e.g., lying down on their back supine). The dashed line 1002 corresponds to the distance between the button electrodes (e.g., 108a) of the aLP and the vLP, which distance can also be referred to as the aLPbutton-vLPbutton distance. The dashed line 1004 corresponds to the distance between centers of the aLP and vLP, which distance can be referred to as the aLPcenter-vLPcenter distance. The angle ϕ corresponds to a vLP-to-aLP angle in one projection plane of view. FIG. 10C is a graph that plots the aLPbutton-vLPbutton distance in millimeters (mm), the aLPcenter-vLPcenter distance in mm, and the vLP-to-aLP angle ϕ in degrees, at various different times (also referred to as frames) throughout a cardiac cycle (e.g., there can be 15 frames per second). The aLPcenter-vLPcenter distance in FIG. 10C is represented by small circles and varies between about 74 mm and 82 mm. The aLPbutton-vLPbutton distance in FIG. 10C is represented by small triangles and varies between about 53 mm and 65 mm. The vLP-to-aLP angle in FIG. 10C is represented by small squares and varies between about 36 degrees and 79 degrees. As can be appreciated from FIG. 10C, the vLP-to-aLP angle ϕ, which is indicative of the orientation of the aLP and vLP relative to one another, varies the most throughout the cardiac cycle for which data is plotted.

Embodiments of the present technology described herein improve and preferably optimize the timing of conducted i2i communication to take advantage of the decrease in ventricular volume that occurs during mechanical contraction and the accompanying twisting motion causing shortening of the heart anatomical axis. Such embodiments identify an alternate or backup time window (i.e., the ACW) for conducted i2i communication with more favorable throughput performance while reducing the risk of inadvertent capture of partially repolarized tissue during conducted i2i communication.

In the exemplary LP 102, 104 described above with reference to FIGS. 1A, 1B and 2, the LP was shown as and described as having a pair of electrodes 108 (e.g., labeled 108a and 108b in FIG. 2). As was mentioned above, one of the electrodes 108 (e.g., 108a) can function as a cathode type electrode and another one of the electrodes 108 (e.g., 108b) can function as an anode type electrode, or vice versa, when the electrodes are used for delivering stimulation. In FIG. 2, the electrode 108b is shown as being a ring electrode that extends around an entire circumference of a portion of the LP 102, 104. In alternative embodiments, the electrode 108b can be a slit ring electrode, meaning the electrode 108b can be capable of being electrically separated into halves of some other portions (e.g., thirds, fourths, etc.), depending upon design. For example, if split into halves, each half can take up 180 degrees of a 360 degree ring; or if slit into quarters, each quarter can take up 90 degrees of a 360 degree ring. By changing which one or more portion(s) of the electrode 108b is/are used for sensing (or transmitting) communication pulses, the sense vector (or transmission vector) can be adjusted to improve conducted communication in dependence on the relative orientation and distance of two LPs (or other types of IMDs) as determined based on accelerometer outputs, and/or based on where the LPs are within a cardiac cycle as determined based on an IEGM. More generally, in accordance with certain embodiments, electrode sensing and/or transmitting vectors can be adjusted based on one or more accelerometer outputs and/or an IEGM to improve conducted communication.

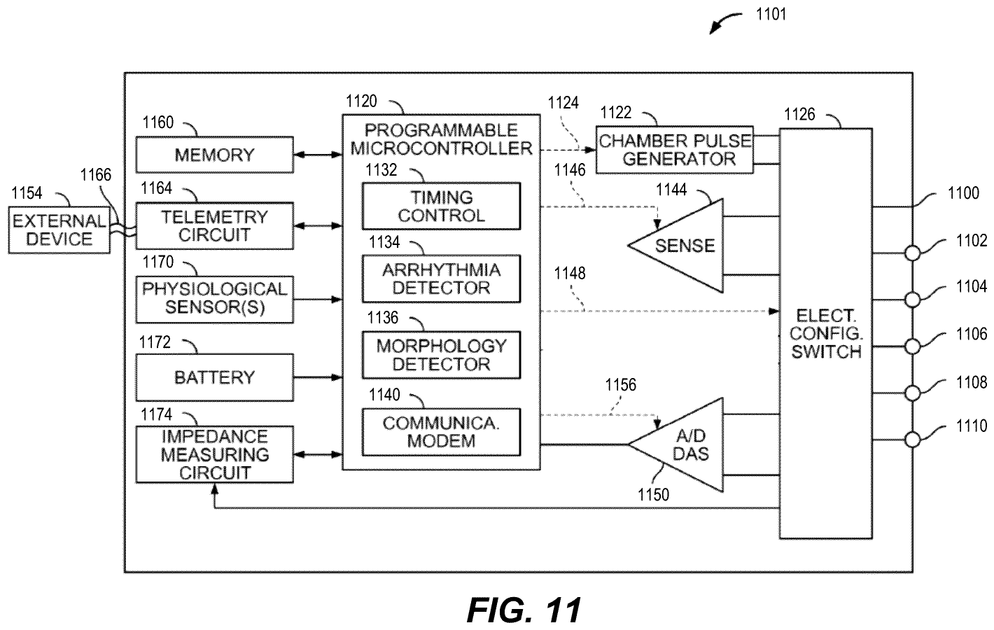

FIG. 11 shows a block diagram of one embodiment of an LP 1101 (or other type of IMD) that is implanted into the patient as part of the implantable cardiac system in accordance with certain embodiments herein. LP 1101 may be implemented as a full-function biventricular pacemaker, equipped with both atrial and ventricular sensing and pacing circuitry for four chamber sensing and stimulation therapy (including both pacing and shock treatment). Optionally, LP 1101 may provide full-function cardiac resynchronization therapy. Alternatively, LP 1101 may be implemented with a reduced set of functions and components.

LP 1101 has a housing 1100 to hold the electronic/computing components. Housing 1100 (which is often referred to as the “can”, “case”, “encasing”, or “case electrode”) may be programmably selected to act as the return electrode for certain stimulus modes. Housing 1100 may further include a connector (not shown) with a plurality of terminals 1102, 1104, 1106, 1108, and 1110. The terminals may be connected to electrodes that are located in various locations on housing 1100 or elsewhere within and about the heart. LP 1101 includes a programmable microcontroller 1120 that controls various operations of LP 1101, including cardiac monitoring and stimulation therapy. Microcontroller 1120 includes a microprocessor (or equivalent control circuitry), RAM and/or ROM memory, logic and timing circuitry, state machine circuitry, and I/O circuitry.

LP 1101 further includes a pulse generator 1122 that generates stimulation pulses and communication pulses for delivery by one or more electrodes coupled thereto. Pulse generator 1122 is controlled by microcontroller 1120 via control signal 1124. Pulse generator 1122 may be coupled to the select electrode(s) via an electrode configuration switch 1126, which includes multiple switches for connecting the desired electrodes to the appropriate I/O circuits, thereby facilitating electrode programmability. Switch 1126 is controlled by a control signal 1128 from microcontroller 1120.

In the embodiment of FIG. 11, a single pulse generator 1122 is illustrated. Optionally, the IMD may include multiple pulse generators, similar to pulse generator 1122, where each pulse generator is coupled to one or more electrodes and controlled by microcontroller 1120 to deliver select stimulus pulse(s) to the corresponding one or more electrodes.

Microcontroller 1120 is illustrated as including timing control circuitry 1132 to control the timing of the stimulation pulses (e.g., pacing rate, atrio-ventricular (AV) delay, atrial interconduction (A-A) delay, or ventricular interconduction (V-V) delay, etc.). Timing control circuitry 1132 may also be used for the timing of refractory periods, blanking intervals, noise detection windows, evoked response windows, alert intervals, marker channel timing, and so on. Microcontroller 1120 also has an arrhythmia detector 1134 for detecting arrhythmia conditions and a morphology detector 1136. Although not shown, the microcontroller 1120 may further include other dedicated circuitry and/or firmware/software components that assist in monitoring various conditions of the patient's heart and managing pacing therapies. The microcontroller can include a processor. The microcontroller, and/or the processor thereof, can be used to perform the methods of the present technology described herein.

LP 1101 is further equipped with a communication modem (modulator/demodulator) 1140 to enable wireless communication with the remote slave pacing unit. Modem 1140 may include one or more transmitters and two or more receivers as discussed herein in connection with FIG. 1B. In one implementation, modem 1140 may use low or high frequency modulation. As one example, modem 1140 may transmit i2i messages and other signals through conducted communication between a pair of electrodes. Modem 1140 may be implemented in hardware as part of microcontroller 1120, or as software/firmware instructions programmed into and executed by microcontroller 1120. Alternatively, modem 1140 may reside separately from the microcontroller as a standalone component.

LP 1101 includes a sensing circuit 1144 selectively coupled to one or more electrodes, that perform sensing operations, through switch 1126 to detect the presence of cardiac activity in the right chambers of the heart. Sensing circuit 1144 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the unit to sense low amplitude signal characteristics of atrial fibrillation. Switch 1126 determines the sensing polarity of the cardiac signal by selectively closing the appropriate switches. In this way, the clinician may program the sensing polarity independent of the stimulation polarity.

The output of sensing circuit 1144 is connected to microcontroller 1120 which, in turn, triggers or inhibits the pulse generator 1122 in response to the presence or absence of cardiac activity. Sensing circuit 1144 receives a control signal 1146 from microcontroller 1120 for purposes of controlling the gain, threshold, polarization charge removal circuitry (not shown), and the timing of any blocking circuitry (not shown) coupled to the inputs of the sensing circuitry.

In the embodiment of FIG. 11, a single sensing circuit 1144 is illustrated. Optionally, the IMD may include multiple sensing circuits, similar to sensing circuit 1144, where each sensing circuit is coupled to one or more electrodes and controlled by microcontroller 1120 to sense electrical activity detected at the corresponding one or more electrodes. Sensing circuit 1144 may operate in a unipolar sensing configuration or in a bipolar sensing configuration.

LP 1101 further includes an analog-to-digital (A/D) data acquisition system (DAS) 1150 coupled to one or more electrodes via switch 1126 to sample cardiac signals across any pair of desired electrodes. Data acquisition system 1150 is configured to acquire intracardiac electrogram signals, convert the raw analog data into digital data, and store the digital data for later processing and/or telemetric transmission to an external device 1154 (e.g., a programmer, local transceiver, or a diagnostic system analyzer). Data acquisition system 1150 is controlled by a control signal 1156 from the microcontroller 1120.

Microcontroller 1120 is coupled to a memory 1160 by a suitable data/address bus. The programmable operating parameters used by microcontroller 1120 are stored in memory 1160 and used to customize the operation of LP 1101 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart within each respective tier of therapy. The memory 1160 can also be used to store information that specifies a primary communication window (PCW) for performing the conducted communication with another LP when using a primary conducted communication protocol (PCCP), and also specifies an alternate communication window (ACW) for performing conducted communication with the other LP when using an alternate conducted communication protocol (ACCP).

The operating parameters of LP 1101 may be non-invasively programmed into memory 1160 through a telemetry circuit 1164 in telemetric communication via communication link 1166 with external device 1154. Telemetry circuit 1164 allows intracardiac electrograms and status information relating to the operation of LP 1101 (as contained in microcontroller 1120 or memory 1160) to be sent to external device 1154 through communication link 1166.

LP 1101 can further include magnet detection circuitry (not shown), coupled to microcontroller 1120, to detect when a magnet is placed over the unit. A magnet may be used by a clinician to perform various test functions of LP 1101 and/or to signal microcontroller 1120 that external device 1154 is in place to receive or transmit data to microcontroller 1120 through telemetry circuits 1164.

LP 1101 can further include one or more physiological sensors 1170. Such sensors are commonly referred to as “rate-responsive” sensors because they are typically used to adjust pacing stimulation rates according to the exercise state of the patient. However, physiological sensor 1170 may further be used to detect changes in cardiac output, changes in the physiological condition of the heart, or diurnal changes in activity (e.g., detecting sleep and wake states). Signals generated by physiological sensors 1170 are passed to microcontroller 1120 for analysis. Microcontroller 1120 responds by adjusting the various pacing parameters (such as rate, AV Delay, V-V Delay, etc.) at which the atrial and ventricular pacing pulses are administered. While shown as being included within LP 1101, physiological sensor(s) 1170 may be external to LP 1101, yet still be implanted within or carried by the patient. Examples of physiologic sensors might include sensors that, for example, sense respiration rate, pH of blood, ventricular gradient, activity, position/posture, minute ventilation (MV), and so forth.

A battery 1172 provides operating power to all of the components in LP 1101. Battery 1172 is capable of operating at low current drains for long periods of time, and is capable of providing high-current pulses (for capacitor charging) when the patient requires a shock pulse (e.g., in excess of 2 A, at voltages above 2 V, for periods of 10 seconds or more). Battery 1172 also desirably has a predictable discharge characteristic so that elective replacement time can be detected. As one example, LP 1101 employs lithium/silver vanadium oxide batteries.

LP 1101 further includes an impedance measuring circuit 1174, which can be used for many things, including: lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves; and so forth. Impedance measuring circuit 1174 is coupled to switch 1126 so that any desired electrode may be used.

In some embodiments, the LPs 102 and 104 are configured to be implantable in any chamber of the heart, namely either atrium (RA, LA) or either ventricle (RV, LV). Furthermore, for dual-chamber configurations, multiple LPs may be co-implanted (e.g., one in the RA and one in the RV, one in the RV and one in the coronary sinus proximate the LV). Certain pacemaker parameters and functions depend on (or assume) knowledge of the chamber in which the pacemaker is implanted (and thus with which the LP is interacting; e.g., pacing and/or sensing). Some non-limiting examples include: sensing sensitivity, an evoked response algorithm, use of AF suppression in a local chamber, blanking & refractory periods, etc. Accordingly, each LP needs to know an identity of the chamber in which the LP is implanted, and processes may be implemented to automatically identify a local chamber associated with each LP.

Processes for chamber identification may also be applied to subcutaneous pacemakers, ICDs, with leads and the like. A device with one or more implanted leads, identification and/or confirmation of the chamber into which the lead was implanted could be useful in several pertinent scenarios. For example, for a DR or CRT device, automatic identification and confirmation could mitigate against the possibility of the clinician inadvertently placing the V lead into the A port of the implantable medical device, and vice-versa. As another example, for an SR device, automatic identification of implanted chamber could enable the device and/or programmer to select and present the proper subset of pacing modes (e.g., AAI or VVI), and for the IPG to utilize the proper set of settings and algorithms (e.g., V-AutoCapture vs ACap-Confirm, sensing sensitivities, etc.).

While many of the embodiments of the present technology described above have been described as being for use with LP type IMDs, embodiments of the present technology that are for use in improving conducted communication can also be used with other types of IMDs besides an LP. Accordingly, unless specifically limited to use with an LP, the claims should not be limited to use with LP type IMDs. For example, embodiments of the present technology can also be used with a subcutaneous-ICD and/or a subcutaneous pacemaker, but are not limited thereto. For example, where an LP is configured to communicate with an ICD using conducted i2i communication, the embodiments described herein can be used to improve the conducted i2i communication between the LP and the ICD.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, it is noted that the term “based on” as used herein, unless stated otherwise, should be interpreted as meaning based at least in part on, meaning there can be one or more additional factors upon which a decision or the like is made. For example, if a decision is based on the results of a comparison, that decision can also be based on one or more other factors in addition to being based on results of the comparison.

Embodiments of the present technology have been described above with the aid of functional building blocks illustrating the performance of specified functions and relationships thereof. The boundaries of these functional building blocks have often been defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in FIGS. 6, 8 and 9. For another example, it is possible to change the boundaries of some of the dashed blocks shown in FIGS. 1B and 11.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the embodiments of the present technology without departing from its scope. While the dimensions, types of materials and coatings described herein are intended to define the parameters of the embodiments of the present technology, they are by no means limiting and are exemplary embodiments. Many other embodiments will be apparent to those of skill in the art upon reviewing the above description. The scope of the embodiments of the present technology should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112 (f), unless and until such claim limitations expressly use the phrase “means for” followed by a statement of function void of further structure.

What is claimed is:

1. A method for use by a system including first and second implantable medical devices (IMDs) that are configured to perform conducted communication with one another, wherein at least one of the first and second IMDs comprises a leadless pacemaker (LP) configured to be implanted within or on a cardiac chamber, the method comprising:
the first and second IMDs performing the conducted communication with one another during a primary communication window (PCW) of one or more cardiac cycles in accordance with a primary conducted communication protocol (PCCP);
at least one of the first and second IMDs monitoring a quality metric of the conducted communication, while the first and second IMDs are performing the conducted communication with one another in accordance with the PCCP; and
in response to the quality metric of the conducted communication falling below a corresponding threshold, the first and second IMDs performing the conducted communication with one another during an alternative communication window (ACW) of one or more further cardiac cycles in accordance with an alternative conducted communication protocol (ACCP).

2. The method of claim 1, further comprising:
storing respective information within each of the first and second IMDs that specifies the PCW for performing the conducted communication with one another when using the PCCP, and also specifies the ACW for performing the conducted communication with one another when using the ACCP.

3. The method of claim 1, further comprising:
testing a plurality of different windows for performing conducted communication between the first and second LPs to thereby identify the ACW.

4. The method of claim 1, wherein:
a distance between the first and second IMDs and an orientation of the first and second IMDs relative to one another vary over a cardiac cycle; and
the ACW corresponds to a period within the cardiac cycle when the distance between the first and second IMDs and the orientation of the first and second IMDs relative to one another are such that it is likely that the conducted communication between the first and second IMDs will be successful.

5. The method of claim 1, wherein when the first and second IMDs perform conducted communication with one another using the PCCP, the first IMD transmits one or more conducted communication pulses to the second IMD within the PCW that occurs immediately prior to a paced event caused by the first IMD or immediately following a sensed event sensed by the first IMD.

6. The method of claim 1, wherein when the first and second IMDs perform conducted communication with one another during the ACW using the ACCP, the first and second IMDs are also attempting to perform the conducted communication with one another during the PCW.

7. The method of claim 1, wherein when the first and second IMDs perform conducted communication with one another during the ACW using the ACCP, the first and second IMDs do not also attempt to perform the conducted communication with one another during the PCW.

8. The method of claim 1, wherein the performing conducted communication comprises one of the first and second IMDs transmitting one or more conducted communication pulses to the other one of the first and second IMDs, and the other one of the first and second IMDs attempting to receive the one or more conducted communication pulses, and wherein the quality metric is based on one or more of the following:
a measure of amplitude of at least one of the one or more conducted communication pulses received by one of the first and second IMDs;
a magnitude of at least one of the one or more conducted communication pulses received by one of the first and second IMDs;
a measure of signal throughput;
a measure of signal loss in at least one of the one or more conducted communication pulses received by one of the first and second IMDs;
a signal-to-noise ratio (SNR) of at least one of the one or more conducted communication pulses received by one of the first and second IMDs;
a total energy of at one of the one or more conducted communication pulses received by one of the first and second IMDs; or
a bit-error-rate (BER) associated with at least one of the one or more conducted communication pulses received by one of the first and second IMDs.

9. The method of claim 1, wherein the first and second IMDs comprise first and second LPs, and wherein:
one of the first and second LPs is configured to be implanted in or on an atrial cardiac chamber, and the other one of the first and second LPs is configured to be implanted in or on a ventricular cardiac chamber; or
one of the first and second LPs is configured to be implanted in or on a right ventricular cardiac chamber, and the other one of the first and second LPs is configured to be implanted in or on a left ventricular cardiac chamber.

10. The method of claim 1, further comprising:
whenever a critical message is sent from one of the first and second IMDs to the other using conducted communication, the critical message is sent during both the PCW and the ACW of a same cardiac cycle to thereby increase a probability that the critical message will be successful received.

11. The method of claim 1, further comprising, in response to the quality metric of the conducted communication falling below the corresponding threshold, at least one of the IMDs selecting the ACW from a plurality of possible ACWs based on a posture of a patient within which the first and second IMDs are implanted.

12. An implantable system, comprising:
first and second implantable medical devices (IMDs) at least one of which comprises a leadless pacemaker (LP) configured to be implanted in or on a first cardiac chamber;
each IMD, of the first and second IMDs, comprising a respective controller, a respective memory, one or more respective sense circuits, one or more respective pulse generators, and a respective pair of electrodes;
the one or more pulse generators and the pair of electrodes, of at least the LP, configured to output pacing pulses and output conducted communication pulses;
the one or more sense circuits and the pair of electrodes, of each IMD, of the first and second IMDs, configured to sense a cardiac electrical signal and sense conducted communication pulses;
the controller of each IMD, of the first and second IMDs, communicatively coupled to the memory, the one or more sense circuits, and the one or more pulse generators, and configured to perform conducted communication with the other IMD during a primary communication window (PCW) of one or more cardiac cycles in accordance with a primary conducted communication protocol (PCCP);
the controller of at least one of the first and second IMDs configured to
monitor a quality metric of the conducted communication, while the first and second IMDs are performing the conducted communication with one another in accordance with the PCCP; and
in response to the quality metric of the conducted communication falling below a corresponding threshold, cause the conducted communication to be performed with the other IMD during an alternative communication window (ACW) of one or more further cardiac cycles in accordance with an alternative conducted communication protocol (ACCP).

13. The system of claim 12, wherein:
the memory of each IMD of the first and second IMDs stores information that specifies the PCW for performing conducted communication using the PCCP, and also specifies the ACW for performing the conducted communication using the ACCP.

14. The system of claim 12, wherein:
a distance between the first and second IMDs and an orientation of the first and second IMDs relative to one another vary over a cardiac cycle; and
the ACW corresponds to a period within the cardiac cycle when the distance between the first and second IMDs and the orientation of the first and second IMDs relative to one another are such that it is likely that the conducted communication between the first and second IMDs will be successful.

15. The system of claim 12, wherein a plurality of different windows for performing conducted communication between the first and second IMDs are tested to thereby identify the ACW.

16. The system of claim 12, wherein when the first and second IMDs perform conducted communication with one another using the PCCP, the first IMD transmits one or more conducted communication pulses to the second IMD within the PCW that occurs immediately prior to a paced event caused by the first IMD or immediately following a sensed event sensed by the first IMD.

17. The system of claim 12, wherein when the first and second IMDs perform conducted communication with one another during the ACW using the ACCP, the first and second IMDs are also attempting to perform the conducted communication with one another during the PCW.

18. The system of claim 12, wherein when the first and second IMDs perform conducted communication with one another using the ACCP, the first and second IMDs do not attempt to perform conducted communication with one another during the PCW.

19. The system of claim 12, wherein the quality metric is based on one or more of the following:
a measure of amplitude of at least one of the one or more conducted communication pulses received by one of the first and second IMDs;
a magnitude of at least one of the one or more conducted communication pulses received by one of the first and second IMDs;
a measure of signal throughput;
a measure of signal loss in at least one of the one or more conducted communication pulses received by one of the first and second IMDs;
a signal-to-noise ratio (SNR) of at least one of the one or more conducted communication pulses received by one of the first and second IMDs;
a total energy of at one of the one or more conducted communication pulses received by one of the first and second IMDs; or
a bit-error-rate (BER) associated with at least one of the one or more conducted communication pulses received by one of the first and second IMDs.

20. The system of claim 12, wherein the first and second IMDs comprise first and second LPs, and wherein:
one of the first and second LPs is configured to be implanted in or on an atrial cardiac chamber, and the other one of the first and second LPs is configured to be implanted in or on a ventricular cardiac chamber; or
one of the first and second LPs is configured to be implanted in or on a right ventricular cardiac chamber, and the other one of the first and second LPs is configured to be implanted in or on a left ventricular cardiac chamber.

21. The system of claim 12, wherein the controller of at least one of the first and second IMDs is configured such that whenever a critical message is sent from one of the first and second IMDs to the other using conducted communication, the critical message is sent during both the PCW and the ACW of a same cardiac cycle to thereby increase a probability that the critical message will be successful received.

22. The system of claim 12, wherein the controller of at least one of the first and second IMDs is configured to, in response to the quality metric of the conducted communication falling below the corresponding threshold, select the ACW from a plurality of possible ACWs based on a posture of a patient within which the first and second IMDs are implanted.

23. A method for use by a system including first and second implantable medical devices (IMDs) that are configured to perform conducted communication with one another, wherein at least one of the first and second IMDs comprises a leadless pacemaker (LP) configured to be implanted within or on a cardiac chamber, the method comprising:
(a) testing a plurality of different communication windows (CWs) for performing the conducted communication between the first and second IMDs, for each of a plurality of different postures of a patient, to thereby perform the testing for each of a plurality of different combinations of the different postures and the different CWs;
(b) determining and storing information indicative of a respective communication quality metric for each of the plurality of different combinations of the different postures and the different CWs;
(c) selecting one of the CWs to use for performing the conducted communication based on the stored information; and
(d) performing the conducted communication between the first and second IMDs using the selected one of the CWs.

24. The method of claim 23, wherein the selecting comprises selecting the one of the CWs based on the stored information and a posture of the patient.

25. The method of claim 23, wherein each of the plurality of CWs corresponds to a different delay relative to at least one of a paced or sensed event.

26. The method of claim 23, wherein the selecting comprises selecting the one of the CWs that, based on the stored information, should result in the conducted communication being successful regardless of which one of the plurality of different postures the patient is positioned.

27. The method of claim 23, wherein the first and second IMDs comprise first and second LPs, and wherein:
one of the first and second LPs is configured to be implanted in or on an atrial cardiac chamber, and the other one of the first and second LPs is configured to be implanted in or on a ventricular cardiac chamber; or
one of the first and second LPs is configured to be implanted in or on a right ventricular cardiac chamber, and the other one of the first and second LPs is configured to be implanted in or on a left ventricular cardiac chamber.
